(19) United States
(12) Patent Application Publication
(10) Pub. No.: US 2007/0065905 A1
(43) Pub. Date: Mar. 22, 2007

(54) Process for expression and secretion of proteins by the non-conventional yeast zygosaccharomyces bailii

(76) Inventors: Paola Branduardi, Mailand (IT); Danilo Porro, Erba (IT); Minoska Valli, Calcinate (IT); Lllia Alberghina, Mailand (IT)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/534,171

(22) PCT Filed: Nov. 6, 2003
(86) PCT No.: PCT/EP03/12377

§ 371 Date: May 6, 2005

(30) Foreign Application Priority Data
Nov. 7, 2002 (DE) 102 52 245.6

(51) Int. Cl. C12P 21/06; C07H 21/04; C07K 14/47; C12N 15/74; C12N 1/18

(52) U.S. Cl. 435/69.1; 435/483; 435/254.2; 530/350; 536/23.7

(57) ABSTRACT

Herein is disclosed a method for the production of proteins. The protein is expressed by a yeast belonging to the species Zygosaccharomyces bailii. The yeast secretes the protein produced into the culture medium from where it is isolated, thereby simplifying the isolation process. Preferably the yeast is cultivated in chemically defined medium, thereby further simplifying the isolation process significantly.

High level production of proteins from engineered organisms (recombinant, mutagenised, . . . ) provides an alternative to the extraction of the proteins from natural sources. Natural sources of proteins are often limited, and furthermore the concentration of the desired product is generally low so extraction is regularly very cost-intensive or even impossible. Besides, extraction might bear the danger of toxic or infectious contamination depending on the natural origin of the protein.

With the advent of molecular cloning in the mid-70s, it became possible to produce foreign proteins in new hosts. Recombinant DNA (rDNA) technologies (genetic, protein and metabolic engineering) allow the production of a wide range of peptides and proteins from naturally-non producing cells. In fact the first biotech-products on the world market made by means of rDNA were pharmaceutical products (for example insulin, interferons, erythropoietin, vaccine against hepatitis B) and industrial enzymes (for example used for the treatments of food, feed, detergents, paper-pulp and health care). World-sales of the top-20 recombinant pharmaceutical products in 2000 was about 13 billions Euro, while the world-wide market for the industrial enzymes was about 2.0 and it is projected to reach about 8 billions Euro in 2008.

Microorganisms as well as cultured cells from higher organisms (such as mammalians, insects or plants) represent the mainly conceivable hosts for the production of heterologous as well as homologous proteins.

Several processes using mammalian cell culture for the production of proteins have been developed and many in such a manner produced proteins are on the market. Among them, several vaccines, monoclonal antibodies, interferon, blood factors, urokinase and tPA, hormones and growth factors.

The main advantage of a mammalian cell based expression system is the ability of mammalian cells to process the proteins in a proper way (correct folding, appropriate post-translational modification, correct glycosylation, specific proteolytic activities, etc.). A cloned protein expressed from recombinant DNA of mammalian origin (human) is usually correctly processed and folded and commonly secreted into the medium, allowing a fast recovery and purification. On the other hand the costs of production are generally quite high due to a usually low level of expression, costs of the mammalian medium components, very slow growth rates and demanding culture conditions. Furthermore, production in mammalian cells bears the danger of toxic or infectious contamination of the product.

Microorganisms (prokaryotic as well as eukaryotic) are advantageous hosts for the production of proteins because of high growth rates and commonly ease of genetic manipulation. But, in particular, bacterial hosts lack the ability of a correct protein processing and in a lot of cases heterologously produced proteins build up inclusion bodies inside of the bacterial cells, whereupon the proteins are lost, because their enzymatic activity can in most instances not be reconstituted. Due to their incorrect structure any use of such proteins for the treatment of humans is also excluded.

Yeast hosts can combine the advantages of unicellular organisms (i.e., ease of genetic manipulation and growth) with the capability of a protein processing typical for eukaryotic organisms (i.e. protein folding, assembly and post-translational modifications), together with the absence of endotoxins as well as oncogenic or viral DNA. Starting from the early 80s, the majority of recombinant proteins produced in yeast have been expressed using Saccharomyces cerevisiae (Hitzeman, R. A. et al., 1981, Nature 293, 717-22). The choice was determined by the familiarity of molecular biologists to this yeast together with the accumulated knowledge about its genetics and physiology. Furthermore, S. cerevisiae is an organism generally regarded as safe (GRAS). However, this yeast is not an optimal host for the large-scale production of foreign proteins, especially due to its characteristics regarding fermentation needs. In particular, growth of S. cerevisiae shows a very pronounced Crabtree effect, therefore fed-batch fermentation is required to attain high-cell densities (see for example Porro, D., et al., 1991, Res. Microbiol. 142, 535-9). Furthermore, this yeast is comparatively sensitive regarding the culture conditions, for example regarding the pH value and the temperature. Therefore, its cultivation is complicated and requires a highly sophisticated equipment. In addition, the proteins produced by S. cerevisiae are often hyper-glycosylated and retention of the products within the periplasmic space is frequently observed (Reiser, J. et al., 1990, Adv. Biochem. Eng./Biophys. 43, 75-102 and Romanos, M. A. et al., 1992, Yeast 8, 423-88). Furthermore, due to the partial retention of the protein in S. cerevisiae, a fraction of the protein is commonly degraded. These respective degradation products are generally very difficult to remove from the desired product. Disadvantages such as these have promoted a search for alternative hosts, trying to exploit the great biodiversity existing among the yeasts, and starting the development of expression systems in the so-called “non conventional” yeasts. Prominent examples are Hansenula polymorpha (Buckholz, R. G. et al., 1991, Bio/Technology 9, 1067-72); Pichia pastoris (Fleer, R., 1992, Curr. Opin. Biotechnol. 3, 486-96); Kluyveromyces lactis (Gellissen, G. et al., 1997, Gene 190, 87-97); Yarrowia lipolytica (Muller, S. et al., 1998, Yeast 14, 1267-83) among others. Another yeast genus under investigation is the genus Zygosaccharomyces. Eleven species, which appear to be evolutionary quite close to S. cerevisiae and not so far from K. lactis have been classified so far (James, S. A. et al., 1994, Yeast 10, 871-81, Steels, H., et al., 1999, Int. J. Syst. Bacteriol. 49, 319-27 and Kurtzman, C. P., et al., 2001, FEMS Yeast research 1, 133-8). An exceptional resistance to several stresses renders some of the Zygosaccharomyces species potentially interesting for industrial purposes. For example Z. rouxii is known to be salt tolerant (osmophilic) and Z. bailii is known to tolerate high sugar concentrations and acidic environments as well as relatively high temperatures of growth (Makdesi, A. K. et al. 1996, Int. J. Food Microbiol. 33, 169-81 and Sousa, M. J. et al., 1996, Appl. Environm. Microbiol. 62, 3152-7). However, the data available related to the molecular biology of these yeasts are very poor. While expression and secretion of a heterologous protein could be achieved in Z. rouxii (Ogawa, Y. et al. 1990, Agric. Biol. Chem. 54, 2521-9), for Z. bailii just the first molecular tools to successfully transform this yeast and to express heterologous proteins intracellulary have been developed (WO 00/41477). Since purification of intracellular proteins is very elaborate, the use of this host for industrial production processes remains limited. Furthermore, while a lot of such non-conventional yeasts show specific advantages regarding their cultivation requirements, a lot of times these advantages are foiled by unexpected negative characteristics or unsolvable problems in their handling. In a lot of instances the tools for transformation of the organisms or expression of heterologous genes are not developed or the development fails due to unfavourable natural properties of the organism in question. The secretory capabilities often impose further problems for the production of proteins in industrial scale. If the organism does not allow the efficient secretion of the desired protein, the isolation of the product is significantly complicated. In addition, some very interesting products, such as Interleukin 1-β, turned out to be toxic for the cells as long as they are intracellulary located (Fleer, R et al., 1991, Gene 107, 285-95). Production of such proteins is therefore only possible if the host comprises a highly potent secretory system that can be exploited. Another problem come from a potentially different codon usage or codon frequency that can hamper the expression of heterologous genes in such organisms decisively.

In consideration of the state of the art, the problem to be solved by the present invention was to provide a new, easy and economical method for the production of proteins. Apart of being cost effective that method should be easy to perform and allow the production of highly pure proteins in a high yield.

This problem as well as all further not explicitly mentioned problems, that are easily deduced from the introductory explicated contents, are solved by the objects outlined in the claims of the instant invention.

An advantageous process for the production of a protein is provided by a method as outlined in claim one. This method comprises culturing a Zygosaccharomyces bailii strain expressing and secreting the protein and isolating the protein. This process is particularly advantageous in that Z. bailii can be cultured yieldingly in a chemically defined medium without the addition of complex ingredients that have to be separated tediously from the protein produced. Surprisingly, the secretory capacity of this yeast in chemically defined medium is significantly superior to the secretory capacity of S. cerevisiae under identical conditions. A further important advantage is the surprising fact that the protein produced by Z. bailii is not only readily secreted but also near to completion, what is not the case for S. cerevisiae under identical conditions. Through efficient secretion of the desired protein by Z. bailii also no degradation of the protein takes place. Subsequently, the purification of the product is significantly simplified.

Further major advantages of Z. bailii as host organism for protein production, and in particular for production of heterologous proteins are a naturally favourable codon usage as deduced from the examples presented herein and the comparatively low demands on the culture conditions. This is in particular due to a high acid and temperature tolerance as well as a weak Crabtree effect allowing the cultivation with a high sugar concentration from the beginning (i.e. batch instead of fed-batch cultivation) and the omission of extremely sophisticated regulations of the culture conditions such as temperature or pH. Accordingly, this method allows a cost effective production of proteins in an easy way even in industrial scale yielding proteins of high purity.

The term “expression” of a protein by a host cell is well known to the skilled artisan. Usually expression of a protein comprises transcription of a DNA sequence into a mRNA sequence followed by translation of the mRNA sequence into the protein. A more detailed description of the process can be found for example in Knippers, R. et al, 1990, Molekulare Genetik, Chapter 3, Georg Thieme Verlag, Stuttgart.

The term “secretion” of a protein as known in the art means translocation of the protein produced, from inside of the cell to outside of the cell, thereby accumulating the protein in the culture medium. A more detailed description of the process can be found for example in Stryer, L., 1991, Biochemie, Chapter 31, Spektrum Akad. Verlag, Heidelberg, Berlin, New York.

The protein produced might be any protein known in the art for which an industrial production is desirable. For example the protein might be useful in the pharmaceutical field, such as medication or vaccine or in pre-clinical or clinical trials among others (examples are growth hormones, tissue plasminogen activator, hepatitis B vaccine, interferones, erythropoietin). The protein produced might also be useful in industry for example in the area of food production (e.g. β-galactosidase, chymosin, amylases, glucoamylase, amylo-glucosidase, invertase) or textile and paper production (proteases, amylases, cellulases, lipases, catalases, etc.). Enzymes are useful among others as detergents (proteases, lipases and surfactants) and their characteristics of stereo-specificity are furthermore exploitable in a wide number of bioconversions, yielding a desired chiral compound. Another promising application of recombinant enzymes that can be produced by the method of the instant invention is the development of biosensors.

The proteins secreted can vary greatly in size (molecular weight). The herein described method functions well for very small proteins (e. g. IL-1β, 17 kDa, see FIG. 5), but also for quite large proteins (e.g. GAA, 67.5 kDa, see FIG. 8a). The secreted proteins may or may not comprise consensus sites for glycosylation. Such consensus sites might occur naturally or might be introduced by genetic engineering. Depending on the intended use of the protein produced it might also be advantageous to remove naturally occurring consensus sites for glycosylation by genetic engineering, thereby preventing for example hyper-glycosylation of the protein. Remarkably, the herein described method leads to proteins that conserve their desired catalytic characteristics after the secretion (e.g. GAA, see FIG. 8a).

In one embodiment of the present invention the Z. bailii strain is transformed with a vector comprising a DNA sequence coding for the protein, functionally linked to a signal sequence leading to the secretion of the protein and further functionally linked to a promoter leading to the expression of the protein.

The term “vector” refers to any agent as such a plasmid, cosmid, virus, phage, or linear or circular single-stranded or double-stranded DNA or RNA molecule, derived from any source that carries nucleic acid sequences into a host cell. Preferably a vector is capable of genomic integration or autonomous replication. Such a vector is capable of introducing a 5′ regulatory sequence or promoter region and a DNA sequence for a selected gene product into a cell in such a manner that the DNA sequence is transcribed into a functional mRNA, which may or may not be translated and therefore expressed. Preferably the vector is an extra-chromosomal plasmid. Such a plasmid comprises preferably an autonomously replicating sequence (ARS) and advantageously a centromeric sequence (CEN) in addition. More preferable the plasmid is a 2μ-like episomal multicopy plasmid. Even more preferably the plasmid is derived from an endogenous episomal plasmid from a Z. bailii strain such as pSB2 (Utatsu, I. et al., 1987, J. Bacteriol. 169, 5537-45) and more preferably from pZB₁ or pZB₅ (see FIG. 9).

The plasmid pZB₅ was extracted from NCYC 1427 and partially sequenced. Accordingly, the plasmid comprises preferably at least 35, more preferably at least 55 and even more preferably at least 75 and even more preferably at least 100 bases from at least one of the sequences selected from the list of SEQ ID No.: 63, SEQ ID No.: 64, SEQ ID No.: 65, SEQ ID No.: 66, SEQ ID No.: 67, SEQ ID No.: 68, SEQ ID No.: 69, SEQ ID No.: 70 or SEQ ID No.: 71.

Yeast multicopy plasmids (also referred to as 2μ or 2 μm-like plasmids) isolated from different yeast genus or species usually show a well conserved structural homology while having a low sequence homology. Some regulatory elements were identified as necessary and sufficient to build a functional multicopy plasmid. These are:

the recombinase promoting amplification of these plasmids, encoded by the FLP gene. (Blanc H., et al., 1979, Mol. Gen. Genet. 176, 335-42 and Broach J. R. et al., 1980, Cell 21, 501-8);

two inverted repeats (IR-sequences);

a single origin of replication (ARS) at the junction between an internal repeat and a unique region of the plasmid (Broach J. R. et al., 1980, Cell 21, 501-8; Brewer B. J. et al., 1987, Cell 51, 463-71; McNeil J. B., et al., 1980, Curr. Genet. 2, 17-25) and

the regulatory proteins REP1/REP2 (in Z. bailii referred to as TFB/TFC), controlling the amplification process, by limiting the recombinase activity in the cell through-mediated repression of FLP gene expression (Broach J. R. et al., 1980, Cell 21, 501-8; Jayaram M. et al., 1983, Cell 34, 95-104).

Within the scope of the instant invention these key elements of the 2μ plasmid are preferably derived from Z. bailii, even more preferably from Z. bailii NCYC1427 or ATCC36947. Particularly preferred these sequences correspond to SEQ ID No.: 71 (IR-ARS), SEQ ID No.: 72 (FLP), SEQ ID No.: 74 (TFB) and SEQ ID No.: 76 (TFC), respectively. The expressed recombinase and the expressed regulatory proteins exhibit preferably the amino acid sequence shown in SEQ ID No.: 73 (FLP), SEQ ID No.: 75 (TFB) and SEQ ID No.: 77 (TFC), respectively. Preferably the plasmid additionally comprises the homologue upstream regions of the FLP and the TFB/TFC genes, in order to obtain an optimal control of the transcription level.

Generally speaking the plasmid preferably comprises sequences for (autonomous) replication, stabilization and/or plasmid copy number control, obtainable from a Z. bailii strain.

Preferably the plasmid is pEZ₁ (see FIG. 9c)

Particularly preferred is the plasmid pEZ₂ (see FIG. 9d). One preferred way to construct pEZ₂ is to amplify the IR/ARS region and the TFC/FLP genes including their homologous promoters by PCR with the oligos

5′-AGAATCAATCATTTAGTGTGGCAGGAG-3′ (SEQ ID NO.: 90) and

5′-TAAAAACTGCCCGCCATATTTCGTC-3′ (SEQ ID NO.: 91, IRAARS),

5′-AGAATGAACTCAGAGTTCTCTCTTG-3′ (SEQ ID NO.: 86) and

5′-CCTATGTCCGAGTTTAGCGAGCTTG-3′(SEQ ID NO.: 85, FLP/TFC)

and to substitute the ARS/CEN cassette from pZ₃ with these amplified products. Another way is to substitute the 2μ-ori sequence from the plasmid p195 with the aforementioned PCR-products.

Advantageously, the vector comprises a selectable marker. The term selectable marker refers to a nucleic acid sequence whose expression confers a phenotype facilitating identification of cells containing the nucleic acid sequence. Selectable markers include those which confer resistance to toxic chemicals (=dominant marker, e.g. G418, hygromycin, formaldehyde, phleomycin or fluoroacetate like reviewed in Van den Berg, M. et al, 1997, Yeast 13, 551-9) or complement an auxotrophy (=auxotrophic marker, e.g. uracil, histidine, leucine, tryptophane). Auxotrophic selection markers can be used for naturally auxotrophic Z. bailii strains or strains that have been rendered auxotrophic by genetical manipulation, in particular by (partial) deletion or mutagenisation of an essential gene, e.g. HIS3 (Branduardi, P., 2002, Yeast 19, 1165-70). As complementing marker sequence the homologous gene from Z. bailii or a heterologous gene might be employed. Auxotrophic markers are preferred since no component has to be added to the medium to keep the selective pressure during the cultivation.

The term “promoter” or “promoter region” refers to a DNA sequence, usually found upstream (5′) to a coding sequence, that controls expression of the coding sequence by controlling production of messenger RNA (mRNA) by providing the recognition site for RNA polymerase and/or other factors necessary for start of transcription at the correct site. The promoter can be derived from any organism. Preferably the promoter is derived from a yeast, even more preferably from Saccharomyces, Kluyveromyces or Zygosaccharomyces and most preferably from Z. rouxii or Z. bailii. The promoter can be constitutive, inducible or repressible. Inducible promoters can be induced by the addition to the medium of an appropriate inducer molecule or by an appropriate change of the chemical or physical growth environment (such as the temperature or pH value), which will be determined by the identity of the promoter. Repressible promoters can be repressed by the addition to the medium of an appropriate repressor molecule or by an appropriate change of the chemical or physical growth environment (such as the temperature or pH value), which will be determined by the identity of the promoter. Constitutive promoters are preferred, as the use of an appropriate repressor or inducer molecule or an appropriate change of the chemical or physical growth environment is not required. Preferably the promoter is selected from the list of: triose-phosphate isomerase (TPI), glyceraldehyde phosphate dehydrogenase (GAPDH), alcohol dehydrogenase 1 (ADH1), phosphoglycerate kinase (PGK), glyceraldehyde-3-phosphate dehydrogenase (GAP), GAL1, GAL10, acid phosphatase (PHO5), cytochrome C-1 (CYC1), copper-binding metallothionein (CUP1) or a-factor mating pheromone precursor (Mfa1) promoter or the hybrid promoters GAL/CYC1, such as GAL1-10/CYC1, GAP/GAL, PGK/GAL, GAP/ADH2, GAP/PHO5 or CYC1/GRE either from S. cerevisiae, Z. rouxii or Z. bailii, but preferred from Z. bailii. Especially preferred promoters are the TPI promoters either from S. cerevisiae corresponding to SEQ ID No.: 78 or Z. bailii corresponding to SEQ ID No.: 79, but particularly preferred is the TPI promoter from Z. bailii (SEQ ID No.: 79). Further particularly preferred promoters are the GAPDH promoters from Z. rouxii (SEQ ID No.: 92) or Z. bailii.

Furthermore the vector comprises preferably a transcriptional terminator sequence following the coding sequence for the desired protein for efficient mRNA 3 end formation. Such a terminator sequence is preferably derived from a yeast, more preferably from Saccharomyces or Zygosaccharomyces, even more preferably from S. cerevisiae or Z. bailii and most preferably from Z. bailii. A preferred example for a terminator sequence comprises the following tripartite consensus sequence: TAG . . . (T-rich) . . . TA(T)GT . . . (AT-rich) . . . TTT. Another preferred example comprises the sequence motif TTTTTATA.

Further the vector comprises a signalling sequence (=leader sequence; upon expression translated into signal peptide or leader peptide). Such sequences lead to the direction of expressed proteins from the cytosol into the culture medium. In other words signal sequences cause the secretion of the proteins and their accumulation in the medium. Signal sequences generally code for a continuous stretch of amino acids, typically 15 to 60 residues long (up to 150), which characteristically include one or more positively charged amino acid(s) followed by a stretch of about 5 to 10 hydrophobic amino acids, which may or may not be interrupted by non-hydrophobic residues. Preferably the signal peptide comprises 15-45 amino acids, even more preferably 15 to 30 amino acids. Even though their amino acid sequences can vary greatly, the signal peptides of all proteins having the same destination in one organism are functionally interchangeable: physical properties, such as hydrophobicity or the pattern of charged amino acids, often appear to be more important in the signal-recognition process than the exact amino acid sequence.

Preferably the DNA sequence coding for the signal peptide is selected from the list of: SEQ ID NO.: 1, SEQ ID NO.: 3, SEQ ID NO.: 5, SEQ ID NO.: 7, SEQ ID NO.: 9, SEQ ID NO.: 11, SEQ ID NO.: 13, SEQ ID NO.: 15, SEQ ID NO.: 17, SEQ ID NO.: 19, SEQ ID NO.: 21, SEQ ID NO.: 23, SEQ ID NO.: 25, SEQ ID NO.: 27, SEQ ID NO.: 29, SEQ ID NO.: 31, SEQ ID NO.: 33, SEQ ID NO.: 35, SEQ ID NO.: 37, SEQ ID NO.: 39, SEQ ID NO.: 41, SEQ ID NO.: 43, SEQ ID NO.: 45, SEQ ID NO.: 47, SEQ ID NO.: 49, SEQ ID NO.: 51, SEQ ID NO.: 53, SEQ ID NO.: 55, SEQ ID NO.: 57, SEQ ID NO.: 59, SEQ ID NO.: 61. Even more preferably the amino acid sequence of the signal peptide is selected from the list of: SEQ ID NO.: 2, SEQ ID NO.: 4, SEQ ID NO.: 6, SEQ ID NO.: 8, SEQ ID NO.: 10, SEQ ID NO.: 12, SEQ ID NO.: 14, SEQ ID NO.: 16, SEQ ID NO.: 18, SEQ ID NO.: 20, SEQ ID NO.: 22, SEQ ID NO.: 24, SEQ ID NO.: 26, SEQ ID NO.: 28, SEQ ID NO.: 30, SEQ ID NO.: 32, SEQ ID NO.: 34, SEQ ID NO.: 36, SEQ ID NO.: 38, SEQ ID NO.: 40, SEQ ID NO.: 42, SEQ ID NO.: 44, SEQ ID NO.: 46, SEQ ID NO.: 48, SEQ ID NO.: 50, SEQ ID NO.: 52, SEQ ID NO.: 54, SEQ ID NO.: 56, SEQ ID NO.: 58, SEQ ID NO.: 60, SEQ ID NO.: 62. Particularly preferred the DNA sequence coding for the signal peptide is selected from the list of SEQ ID NO.: 1, SEQ ID NO.: 3, SEQ ID NO.: 21 or SEQ ID NO.: 35 correspondingly the amino acid sequence of the signal peptide is preferably selected from the list of SEQ ID NO.: 2, SEQ ID NO.: 4, SEQ ID NO.: 22 or SEQ ID NO.: 36.

The signal peptide is preferably removed from the finished protein. This can occur through activity of a specialised signal peptidase. The signal peptidase can be of homologous or heterologous origin. Therefore, the signal peptide comprises preferably a processing site or a cleavage site that allows for recognition by a specific endopeptidase.

In a preferred embodiment of the present invention the Z. bailii strain is transformed with a vector comprising the DNA sequence coding for the protein, functionally linked to the signalling pre-sequence (16 aa) of the alpha-subunit of the K1 killer toxin of K. lactis (Stark M. J. et al., 1986, EMBO J. 5,1995-2002, SEQ ID NO.: 35 (DNA) and SEQ ID NO.: 36 (peptide)) and further functionally linked to the TPI promoter from S. cerevisiae. More preferably the vector is pZ₃kl (FIG. 1b). Even more preferably the Z. bailii strain is transformed with a vector comprising the DNA sequence coding for the protein, functionally linked to the signal sequence of the K1 killer toxin of K. lactis and further functionally linked to the GAPDH promoter from Z. rouxii. Even more preferably the Z. bailii strain is transformed with a vector comprising the DNA sequence coding for the protein, functionally linked to the signal sequence of the K1 killer toxin of K. lactis and further functionally linked to the TPI promoter from Z. bailii. Particularly preferred said vector is derived from pZ₃bT (FIG. 4a).

In another preferred embodiment of the present invention the Z. bailii strain is transformed with a vector comprising the DNA sequence coding for the protein, functionally linked to the signal sequence of the pre-pro α-factor of S. cerevisiae and further functionally linked to the TPI promoter from S. cerevisiae. Preferably the vector is pZ₃ppα (FIG. 1c). Even more preferably the Z. bailii strain is transformed with a vector comprising the DNA sequence coding for the protein, functionally linked to the signal sequence of the pre-pro α-factor of S. cerevisiae and further functionally linked to the GAPDH promoter from Z. rouxii. Even more preferably the Z. bailii strain is transformed with a vector comprising the DNA sequence coding for the protein, functionally linked to the signal sequence of the pre-pro a-factor of S. cerevisiae and further functionally linked to the TPI promoter from Z. bailii. Particularly preferred said vector is derived from pZ₃bT (FIG. 4a).

In yet another preferred embodiment of the present invention the Z. bailii strain is transformed with a vector comprising the DNA sequence coding for the protein, functionally linked to the zygocin killer toxin pre-sequence of Z. bailii (SEQ ID No.: 59) and further functionally linked to a promoter functional in Z. bailii. Preferably said promoter is the TPI promoter from S. cerevisiae. Even more preferably said promoter ist the TPI promoter from Z. bailii. Most preferred is the GAPDH promoter from Z. rouxii.

The DNA sequence coding for the protein can be derived from animal, bacterial, fungal, plant or viral sources, more preferably from metazoan, mammalian or fungal sources. The expressed protein might therefore be homologous or heterologous to Z. bailii.

Any yeast belonging to the species Z. bailii can be used for the production of proteins in the scope of the present invention. In a preferred embodiment of the invention the Z. bailii strain is transformed. “Transformation” refers to a process of introducing an exogenous nucleic acid sequence (of homologous and/or heterologous origin, recombinant or not) into a cell in which that exogenous nucleic acid is incorporated into a chromosome or is capable of autonomous replication. A cell that has undergone transformation, or a descendant of such a cell, is “transformed” or “recombinant”. If the exogenous nucleic acid comprises a coding region encoding a protein and the protein is produced in the transformed yeast such a transformed yeast is functionally transformed. Preferred methods to transform Z. bailii are electroporation, as described in [WO 00/41477], or the chemical LiAc/PEG/ssDNA method as described by Agatep, R. et al., 1998, Technical Tips Online (http://tto.trends.com).

Preferably the Z. bailii strain that is being transformed is selected from the list of: ATCC 36947, ATCC 60483, ATCC 8766, FRR 1292, ISA 1307, NCYC 128, NCYC 563, NCYC 1416, NCYC 1427, NCYC 1766, NRRL Y-2227, NRRL Y-2228, NRRL Y-7239, NRRL Y-7254, NRRL Y-7255, NRRL Y-7256, NRRL Y-7257, NRRL Y-7258, NRRL Y-7259, NRRL Y-7260, NRRL Y-7261, NRRL Y-27164; particularly preferred are ATCC 36947, ATCC 60483, ATCC 8766 and NCYC 1427.

(ATCC: American Type Culture Collection, Manassas Va., USA; FRR: FRR Culture Collection, North Ryde NSW, Australia; ISA: Culture Collection of the Instituto Superior de Agronomia, Lisbon; NCYC: National Collection of Yeast Cultures, Norwich, UK; NRRL: Agricultural Research Service Culture Collection, Peoria Ill., USA).

Within the scope of the present invention the Z. bailii strain can be subjected to a selection process for improved secretion. Screening for and isolation of such a “super-secreting” phenotype can occur before or after transformation of the respective Z. bailii strain.

In a preferred embodiment of the present invention the Z. bailii gene/s homologous to GAS1 from S. cerevisiae are identified and disrupted. GAS1 is one example for the few cases wherein the key molecules involved in the intriguingly complex secretory pathway have been identified. It was possible to influence the whole secretory mechanism modifying the Gas1 expression level in S. cerevisiae (Vai, M., et al., 2000, Appl. Environ. Microbiol. 66, 5477-9) due to a resultant modification of the organisation of the cell wall structure, namely it was demonstrated that gas1 mutants show a “super-secreting” phenotype (Popolo L., et al., 1997, J. Bacteriol. 180, 163-6; Ram A. F. J., et al., 1998, J. Bacteriol. 180, 1418-24).

In another preferred embodiment of the present invention the Z. bailii strain has undergone one or more mutagenisation/selection cycle(s) to obtain super secreting mutants, comprising chemical or physical mutagenesis. Preferably the mutagenisation is caused by orthovanadate. Orthovanadate is a molecule known to affect the glycosylation process and the cell wall construction in S. cerevisiae (Kanik-Ennulat, C. et al., 1990, Mol. Cell. Biol. 10, 898-909). Methods involving orthovanadate mutagenisation to obtain cells with changed cell wall construction/secretory properties that are useful in the scope of the present invention are disclosed in more detail for example for S. cerevisiae (Willsky. G. R., et al., 1985, J. Bacteriol. 164, 611-7) and K. lactis (Uccelletti, D., et al., 1999, Res. Microbiol. 150, 5-12; Uccelletti. D., et al., 2000, Yeast 16, 1161-71).

Culturing techniques and media suitable for yeast are well known in the art. Typically, but it is not limited to, culturing is performed by aqueous fermentation in an appropriate vessel. Examples for a typical vessel for yeast fermentation comprise a shake flask or a bioreactor.

The culture is typically performed at a temperature between 20° C. and 40° C., preferably between 25° C. and 35° C. and even more preferred between 28° C. and 32° C.

The medium in which the Z. bailii strain is cultured can be any medium known in the art to be suitable for this purpose. The medium might contain complex ingredients or might be chemically defined. Chemically defined media are preferred. The medium comprises any component required for the growth of the yeast. In particular the medium comprises a carbon source, such as fructose, glucose or other carbohydrates (such as sucrose, lactose, D-galactose, or hydrolysates of vegetable matter, among others). Typically, the medium also comprises further a nitrogen source, either organic or inorganic, and optionally the medium may also comprise macro nutrients and/or micro nutrients such as amino acids; purines; pyrimidines; corn steep liquor; yeast extract; protein hydrolysates, such as peptone; vitamins (water-soluble and/or water-insoluble), such as B complex vitamins; or inorganic salts such as chlorides, hydrochlorides, phosphates, or sulphates of Ca, Mg, Na, K, Fe, Ni, Co, Cu, Mn, Mo, or Zn, among others. Antifoam might be added, if necessary. Further components known to one of ordinary skill in the art to be useful in yeast culturing or fermentation can also be included. The medium may or may be not buffered. A preferred medium comprises yeast extract, peptone and glucose (=YPD). A more preferred medium comprises yeast extract, peptone and fructose (=YPF). An even more preferred medium comprises glucose and Yeast Nitrogen Base (YNB, Difco Laboratories, Detroit, Mich. #919-15). Another even more preferred medium comprises fructose and YNB.

Particularly preferred is a medium comprising high fructose corn syrup as carbon source (for example Isosweet® 100 42% High Fructose (80% solids) or Isosweet® 5500 55% Fructose from Tate & Lyle PLC or IsoClear® 42% High Fructose Corn Syrup or IsoClear® 55% High Fructose Corn Syrup from Cargill, Inc.).

The compositions of preferred media for batch/fed batch cultivation of Z. bailii according to the instant invention are as follows: the batch phase medium comprises 4% w/V Glucose, 0.5% w/V (NH₄)₂SO₄, 0.05% w/V MgSO₄, 0.3% w/V KH₂PO₄, vitamins according to Verduyn, C., et al., 1992, Yeast 8, 501-17, wherein the final concentration of vitamins will be 3 times in respect to the indicated concentrations and trace elements according to Verduyn, C., et al., 1992, Yeast 8, 501-17, wherein the final concentration will also be 3 times in respect to the indicated concentrations. The pH control (value: pH 5) is performed by the addition of 2M KOH. The fed-batch medium comprises 50% w/V Glucose, 15.708 g/l KH₂PO₄, 5 g/l KCl, 5.831 g/l MgSO₄, 1.2 g/l CaCl₂, 1 g/l Yeast Extract, 0.4447 g/l NaCl, 1 g/l Glutamate, 0.05 g/l ZnSO₄, 0.04 g/l CuSO₄, 0.05 g/l MnCl₂, 0,001 g/l CoCl₂, 0.5 g/l myo-inositol, 0.1 g/l thiamine hydrochloride, 0.02 g/l pyridoxol hydrochloride, 0.04 g/l Ca-D(+)panthotenate, 0.004 g/l d-biotin, 0.09 g/l nicotinic acid. The pH control (value: pH 5) is performed by the addition of 2M NH₄OH.

In case of selection for the dominant G418 marker 200 mg/l G418 is added to the respective medium.

The use of a defined medium, of which the components are adjusted to the needs of the organism is preferred. The purification of the protein is thereby significantly simplified.

Preferably, the pH of the culture medium ranges between 2 and 9, more preferably between 3 and 8 and even more preferably between 4 and 7. The pH can be regulated or partially regulated or not be regulated during the course of fermentation; accordingly the pH can be kept constant at a preferred value or can change during fermentation. A significant advantage of Z. bailii is its surprising capacity to grow as well as express and secrete proteins at low pH. Therefore, the demand of this organisms for a strictly controlled pH is not very pronounced.

The cultivation can take place in batch, fed-batch or continuous mode as is known to the ordinary skilled artisan.

During the course of the fermentation, the desired protein is expressed, properly processed (i.e. folded, modified, cut, etc.) and secreted (=accumulated in the medium). While the protein produced may be partially retained within the yeast cells it is preferred that a substantial amount of the protein is secreted. Even more preferred is that the protein is entirely secreted.

After culturing has progressed for a sufficient length of time to produce a desired concentration of the protein in the yeast and/or the culture medium, the protein is isolated. “Isolated,” as used herein to refer to the protein, means being brought to a state of greater purity by separation of the protein from at least one other component of the yeast or the medium. Preferably, the isolated protein is at least about 80% pure as based on the weight, more preferably at least about 90% pure as based on the weight and even more preferably at least about 95% pure as based on the weight. Evidence of purity can be obtained by SDS-PAGE, 2D electrophoresis, IF, HPLC, mass spectrometry, capillary electrophoresis or other methods well known in the art.

“Purity” refers to the absence of contaminants in the final purified protein. Typical contaminants to be separated from the desired product are proteins, pyrogens, nucleic acids and more.

The protein is isolated from the culture medium, preferably without lysing of the cells. Such an isolation comprises purifying the protein from the medium. Purification can be achieved by techniques well-known in the art, such as filtration (e.g. microfiltration, ultrafiltration, nanofiltration), crystallisation or precipitation, centrifugation, extraction, chromatography (e.g. ion exchange, affinity, hydrophobic exchange), among others.

Upon removal of the cells, the culture broth might also directly serve as the product (e.g. enzyme solution), without further purification. The medium components can be adjusted appropriately prior to the cultivation.

If the protein is not completely secreted, the protein can also be isolated from both the yeast cells and the medium. Methods for lysing of the yeast cells are known in the art and comprise chemical or enzymatic treatment, treatment with glass beads, sonication, freeze/thaw cycling, or other known techniques. The protein can be purified from the various fractions of the yeast lysate by appropriate techniques, such as filtration (e.g. microfiltration, ultrafiltration, nanofiltration), crystallisation or precipitation, centrifugation, extraction, chromatography (e.g. ion exchange, affinity, hydrophobic exchange), among others.

Another embodiment of the present invention relates to a Z. bailii strain, expressing and secreting a heterologous protein.

The Z. bailii strain might be transformed with a vector comprising a DNA sequence coding for the heterologous protein, functionally linked to a signal sequence leading to the secretion of the protein and further functionally linked to a promoter.

### DESCRIPTION OF THE FIGURES

FIG. 1 :Expression and Secretion Vectors

Schematic maps of the plasmids constructed for expression of proteins in Z. bailii: a: pZ₃, (intracellular expression), b: pZ₃kl (expression and secretion) and c: pZ₃ppα (expression and secretion).

a) pZ₃: the backbone of the plasmid is the pYX022 S. cerevisiae expression plasmid (R&D Systems, Inc., Wiesbaden, D; the expression cassette is based on the constitutive S. cerevisiae TPI promoter and the corresponding polyA signal, as indicated in the Figure). The ARS/CEN fragment, from Ycplac33 (Gietz, R. D., et al., 1988, Gene 74, 527-34) ensures replication and stability of the plasmid, while the Kan^{R} cassette, derived from pFA6-KanMX4 (Wach, et al., 1994, Yeast 10, 1793-808) allows a G418-based selection of the transformants.

b) pZ₃kl: a pZ₃ expression vector comprising the signal sequence of the K. lactis K1 killer toxin (kl) for leading the secretion of the protein of interest.

c) pZ3ppa: a pZ₃ expression vector comprising the pre-pro leader sequence of the S. cerevisiae pheromone α-factor (pre-pro-αF) for leading the secretion of the protein of interest.

(Amp=ampicillin resistance cassette; MCS=multi cloning site; colE1 ori; E. coli replication origin)

FIG. 2: Expression and Secretion Vectors

Schematic maps of the plasmids constructed for expression and secretion of the human IL-1β (Auron, E., et al., 1984, PNAS 81, 7907-11) and the GFP (Heim, R. et al., 1996, Curr. Biol. 6, 178-82) in Z. bailii.

a) pZ₃klL-1β: a pZ₃kl vector where the sequence encoding for the human IL-1β was sub-cloned into the MCS.

b) pZ₃ppαIL-1β: a pZ₃ppα vector where the sequence encoding for the human IL-1β was sub-cloned into the MCS.

c) pZ₃ ppαGFP: a pZ₃ppα vector where the sequence encoding for the GFP was sub-cloned into the MCS.

FIG. 3: Expression and Secretion Vectors

Schematic maps of the plasmids constructed for the expression of the Arxula adeninivorans glucoamylase (GAA, Genebank accession no: Z46901, Bui Minh, D., et al., 1996, Appl. Microbiol. Biotechnol. 44, 610-9) and of the bacterial β-galactosidase (from the plasmid pSV-β-galactosidase of Promega, Inc.; Genebank accession no.: X65335) in Z. bailii.

a) pZ₃GAA: a pZ₃ vector where the sequence encoding for the glucoamylase (GAA) was sub-cloned into the MCS.

b) pZ₃LacZ: a pZ₃ vector where the sequence encoding for the β-galactosidase was sub-cloned into the MCS.

FIG. 4: Expression Vectors

Schematic maps of the plasmids constructed for the expression of proteins in Z. bailii based on the Z. bailii TPI promoter.

a) pZ₃bT: a pZ₃ vector where the S. cerevisiae TPI promoter was substituted by the Z. bailii TPI promoter.

b) pZ₃bTLacZ: a pZ₃bT expression vector where the sequence encoding for the β-galactosidase was sub-cloned into the MCS.

FIG. 5: IL-1β secretion

a) Growth kinetics in minimal (YNB) and rich (YPD) medium, with glucose 5% (w/V) as a carbon source: the cellular growth was measured as optical density (OD 660 nm, circles) and the residual glucose (g/l, squares) was evaluated. Comparison between S. cerevisiae (open symbols) and Z. bailii (full symbols).

b) Western Blot analyses performed on cellular extracts of S. cerevisiae and Z. bailii cells transformed with the plasmid pZ₃klIL-1β (expressing IL-1β preceded by the leader sequence from the K. lactis killer toxin) and with the corresponding empty plasmid (PZ₃), as a negative control. In the first lane a positive control (IL-1β, human recombinant (E. coli), Roche cat n^{o} 1 457 756) was loaded. Samples were collected at the indicated times and from the indicated media, corresponding to the kinetics showed in (a). The loaded volumes were rectified for a corresponding OD value of 0.08. The blotted membranes were probed with an α-IL-1β polyclonal antibody.

c) as above, were the loaded samples represent the corresponding supernatant.

d) as above, were the samples were loaded with an equal volume of medium (30 μl).

FIG. 6: Leading of the pre-pro-α-factor signal sequence to the secretion of IL-1β and of GFP in Z. bailii

a) Western Blot analyses performed on cellular extracts (i) and on supernatants (ii) of Z. bailii and S. cerevisiae cells transformed with the plasmid pZ₃ppaIL-1β (and with the corresponding empty plasmid pZ₃) and growing on YPD medium (glucose 2% w/V). Samples were taken at the indicated times. First lane: positive control (IL-1β, human recombinant (E. coli), Roche cat n^{o} 1 457 756). The blotted membranes were probed with an α-IL-1β polyclonal antibody. Western Blot analyses performed on cellular extracts (iii) and on supernatants (iv) of Z. bailii and S. cerevisiae cells transformed with the plasmid pZ₃ppaIL-1β (and with the corresponding empty plasmid pZ₃) and growing on YNB medium (glucose 5% w/V). Samples were taken at indicated times. First lane: positive control (IL-1β human recombinant (E. coli) Roche cat n^{o} 1 457 756). The blotted membranes were probed with an α-IL-1β polyclonal antibody.

b) Western Blot analyses performed on cellular extracts (cells) and on supernatants (sup) of Z. bailii cells growing on YNB medium (glucose 2% w/VV) transformed with the control plasmid pZ₃ (1^{st} and 2^{nd} lanes) and with the plasmid pZ₃ppaGFP (3^{rd} and 4^{th} lanes). The blotted membrane was probed with an α-GFP polyclonal antibody. An arrow indicates the expected positive signal.

FIG. 7: Batch cultivations of Z. bailii cells comprising the pZ₃klIL-1β expression plasmid on chemically defined medium in high sugar concentration

a) Culture OD (full circles), dry weight (open circles), glucose consumption (full squares) and ethanol production (open triangle).

b) Western Blot analyses performed on the growth medium (lane 2 to 5) and on the cell extracts (lanes 6 to 9) of Z. bailii cells. Samples were collected at the indicated times of the kinetics, and an equal volume (30 μl for the supernatants and 15 μl for the cell extracts, respectively) was loaded. The blotted membranes were probed with an α-IL-1β polyclonal antibody.

First lane: positive control (IL-1β human recombinant (E. coli) Roche cat n^{o} 1 457 756).

FIG. 8: Enzymatic activity of heterologous enzymes expressed in Z. bailii cells

a) Determination of the A. adeninivorans glucoamylase activity (mU/OD) present in the growth medium (YNB, glucose 2% w/V) of Z. bailii cells transformed with the plasmid pZ₃GAA (and the respective empty plasmid pZ₃, as a control). Three independent clones were analysed (Cl.1, Cl.3 and Cl.5).

b) Determination of the β-galactosidase activity (Miller U/OD) in cell extracts of Z. bailii cells transformed with the plasmid pZ₃LacZ (two independent clones) and with the plasmid pZ₃bTLacZ (three independent clones), and the respective empty plasmid pZ₃ as a control. Cells were grown in YPD medium (glucose 2% w/V), and samples were collected at indicated times.

On the left panel the Z. bailii strain ATCC 36947, on the right panel the strain Z. bailii ATCC 60483 were tested, respectively.

FIG. 9: Construction of a Z. bailii multicopy plasmid

Schematic maps of the endogenous plasmids isolated from Z. bailii ATCC 36947, named pZB₁ (a) and from Z. bailii NCYC 1427, named pZB₅ (b).

c): Z. bailii multicopy expression vector comprising the genes and the sequences necessary and sufficient for a stable and autonomous high copy number replication. The expression cassette is based on the Z. bailii constitutive TPI promoter and the polyA, as indicated in the Figure. The marker for selection is the Kan^{R} cassette.

d) Z. bailii multicopy expression vector. The expression cassette is based on the Z. bailii constitutive TPI promoter and the polyA, as indicated in the Figure. Furthermore, the vector comprises the IR/ARS region and the TFC/FLP genes including their homologous promoters as indicated.

FIG. 10: Influence of the promoter or the plasmid constituents, respectively, on β-galactosidase activity.

Shown is the relative β-galactosidase activity in cell extracts of Z. bailii ATCC 36947 cells transformed with the indicated plasmids. The β-galactosidase activity of cells transformed with pZ₃LacZ was set to 1 and the other activities were related to that value. Cells were grown in YPD medium (glucose 2% w/V), and samples were collected as the cultures reached an OD⁶⁶⁰ value between 1 and 2.

a) Different promoters in the same plasmid. pZ₃: ScTPI, pZ₃bT: ZbTPI, pZ₃rG: ZrGAPDH.

b) Different plasmid constituents. pZ3: Sc ARS/CEN, p195: Sc 2 μm ori sequence, pEZ-IA: Zb 2 μm ori sequence (IR-A), pEZ-LAF: Zb 2 μm ori sequence (IR-A)+FLP, pEZ₂: Zb 2 μm ori sequence (IR-A)+FLP+TFC, pEZ₂-IB: Zb 2 μm ori sequence (IR-A)+FLP+TFC+IR-B. The table indicates the determined plasmid stability of the respective constructs.

FIG. 11: Leading of the zygocin pre-sequence to the secretion of Il-1β and comparison of different leader sequences

a) Western Blot analyses performed on cellular extracts (i) and on supernatants (ii) of Z. bailii and S. cerevisiae cells transformed with the plasmid pZ₃kbIL-1β (and with the corresponding empty plasmid pZ₃) and growing on YPD medium (glucose 2% w/V). Samples were taken at the indicated times. First lane: positive control (IL-1β, human recombinant (E. coli), Roche cat n^{o} 1 457 756). The blotted membranes were probed with an α-IL-1β polyclonal antibody.

Western Blot analyses performed on cellular extracts (iii) and on supernatants (iv) of Z. bailii and S. cerevisiae cells transformed with the plasmid pZ₃kbIL-1β (and with the corresponding empty plasmid pZ₃) and growing on YNB medium (glucose 5% w/V). Samples were taken at the indicated times. The blotted membranes were probed with an α-IL-1β polyclonal antibody.

b) Western Blot analyses performed on supernatants of Z. bailii cells growing on YNB medium (glucose 2% w/V) transformed with the indicated plasmids. The blotted membranes were probed with an α-IL1β polyclonal antibody.

FIG. 12: Glucoamylase Sta2 activity in transformed Z. bailii or S. cerevisiae cells, respectively

Determination of the S. cerevisiae var. diastaticus glucoamylase Sta2 activity (U/OD) in the growth medium (YNB, fructose 2% w/VV) of Z. bailii and S. cerevisiae cells transformed with the plasmids pZ₃STA2 and pZ₃klSTA2 and the respective empty plasmid pZ₃, as a control (as indicated). In the first plasmid the protein is lead to secretion from its own leader sequence, in the second from the K. lactis killer toxin pre-leader sequence. Measurements were repeated more times and on independent clones, and variation levels are indicated with error bars.

### EXAMPLES

The following examples are included to demonstrate preferred embodiments of the invention. It should be appreciated by those of skill in the art that the techniques disclosed in the examples which follow represent techniques discovered by the inventors to function well in the practice of the instant invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Construction of Z. bailii Expression Plasmids

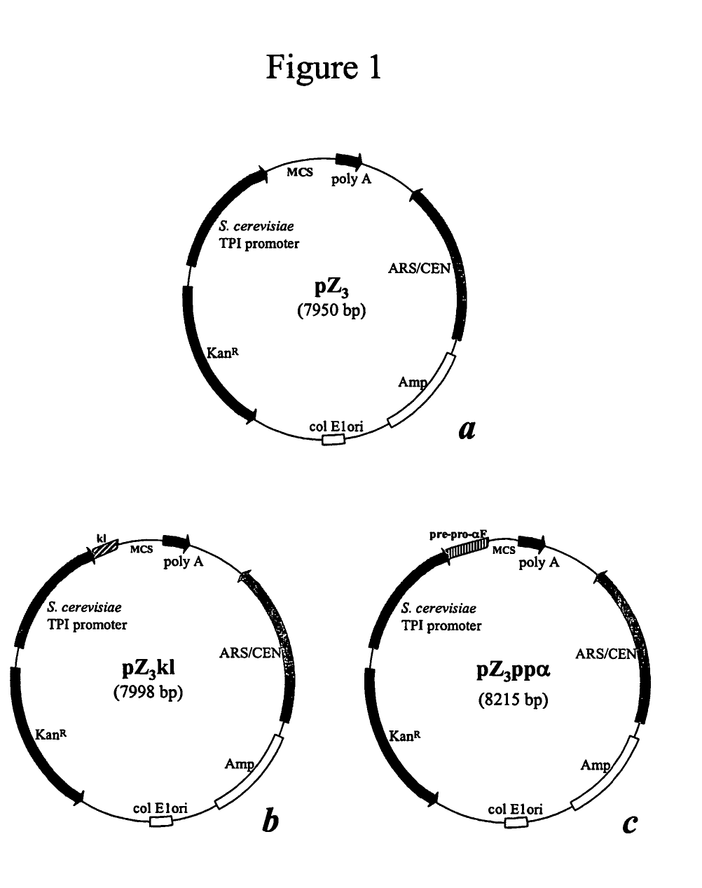

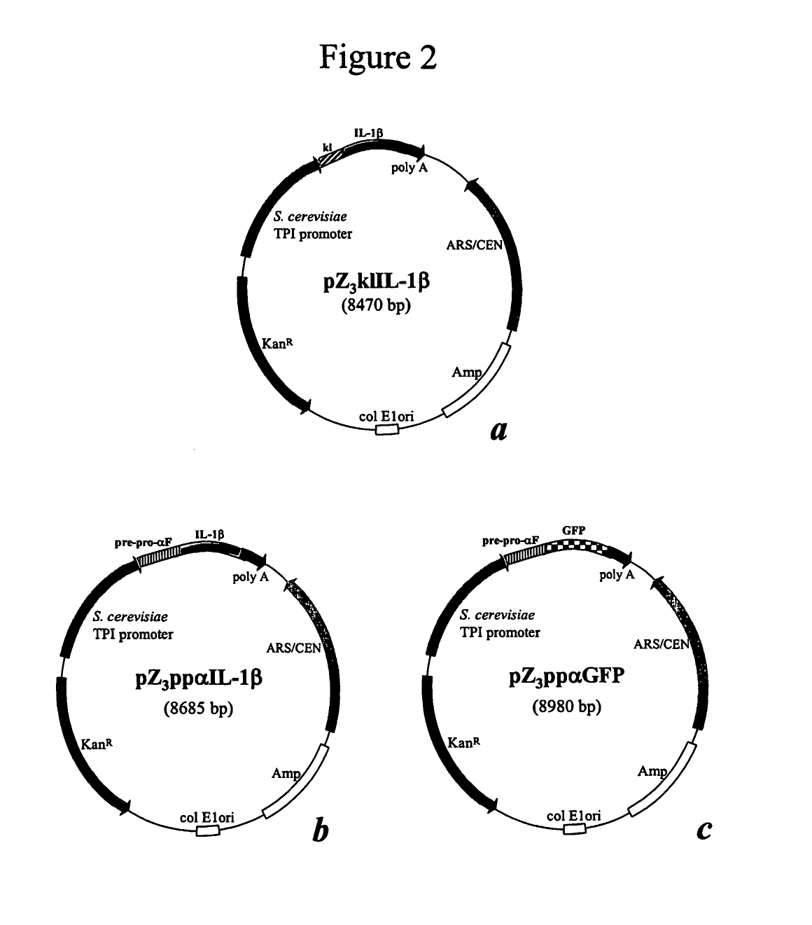

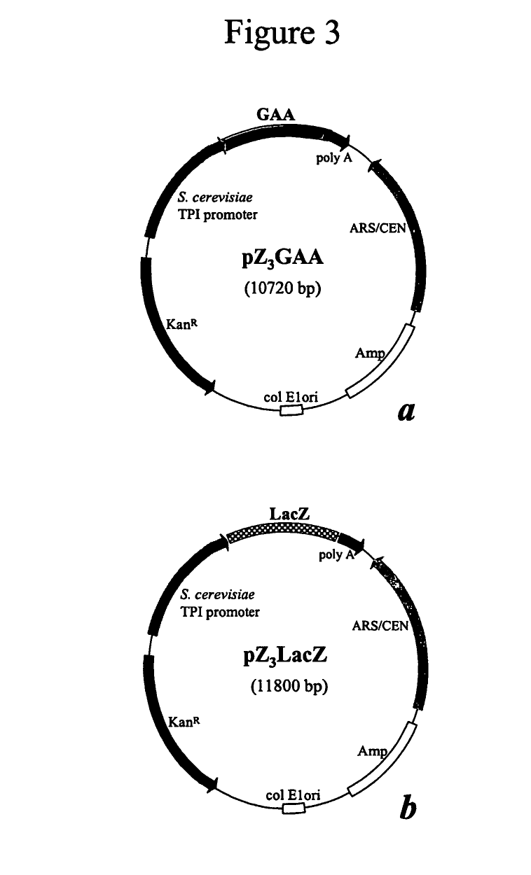

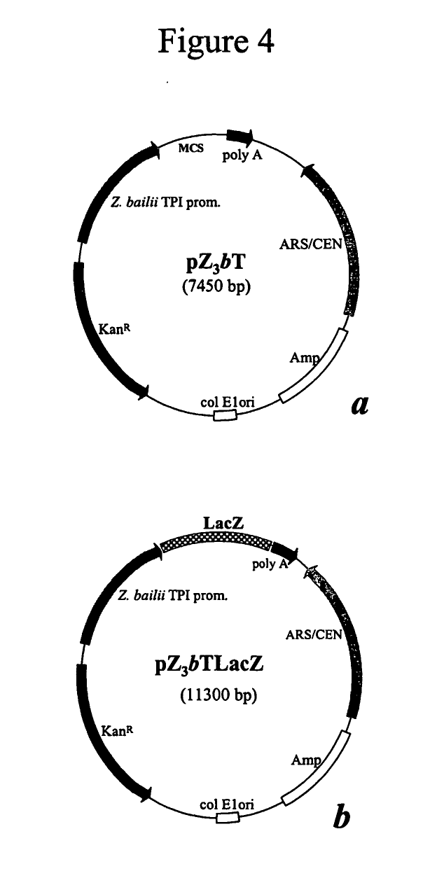

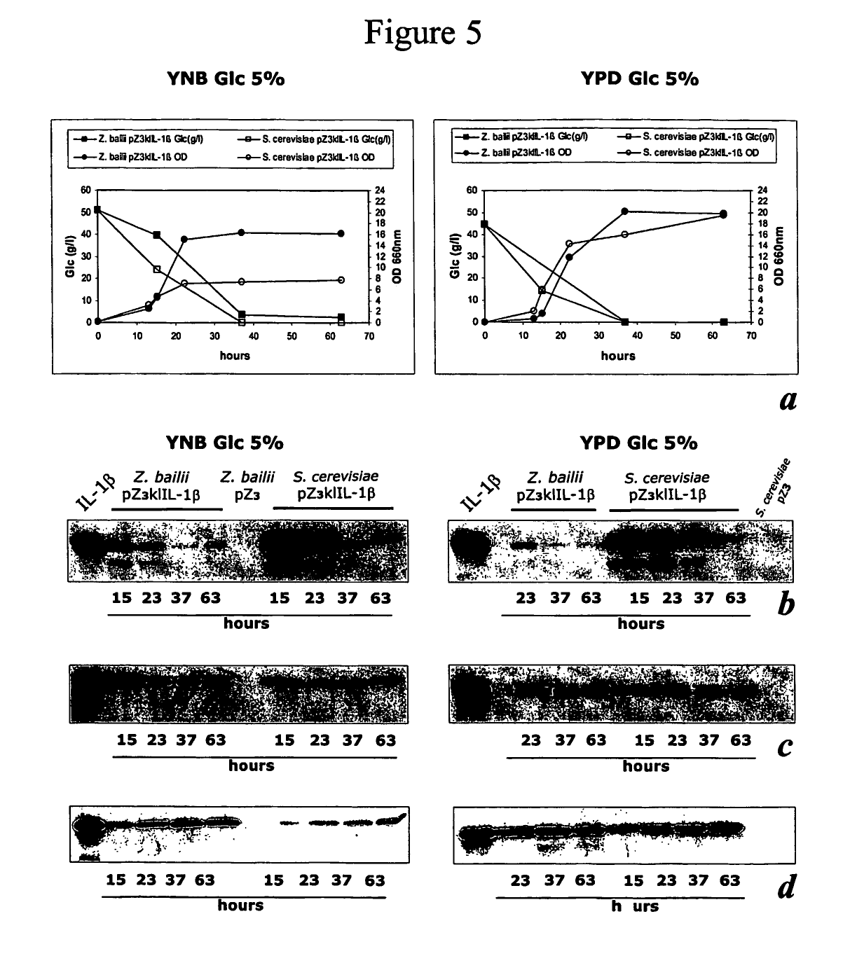

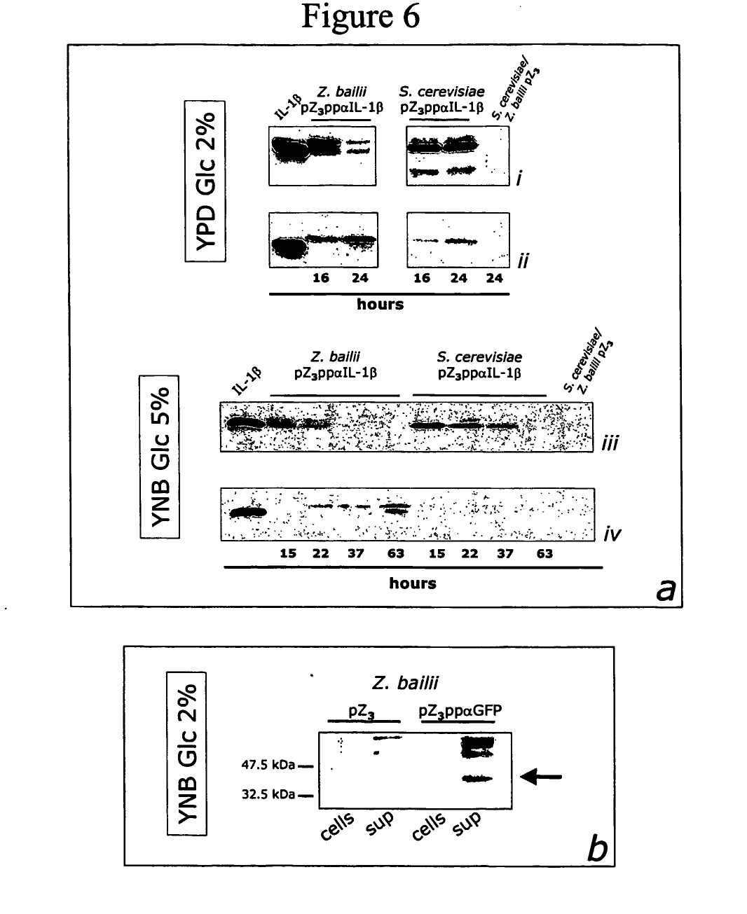

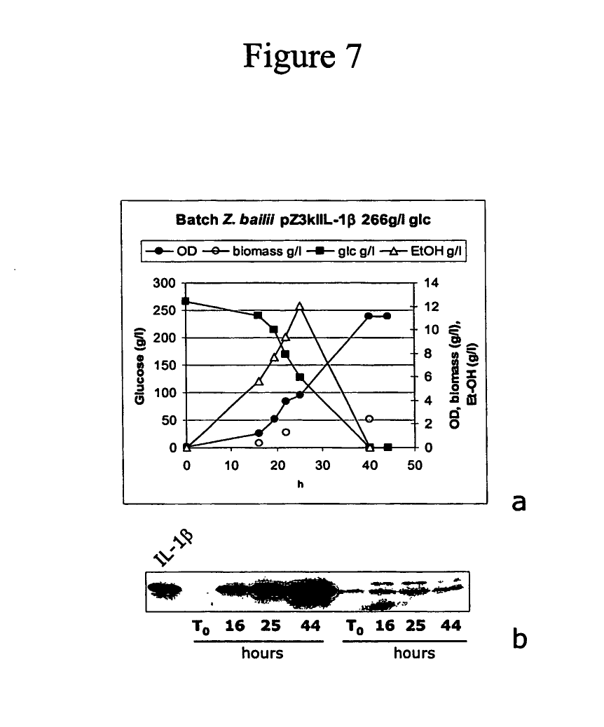

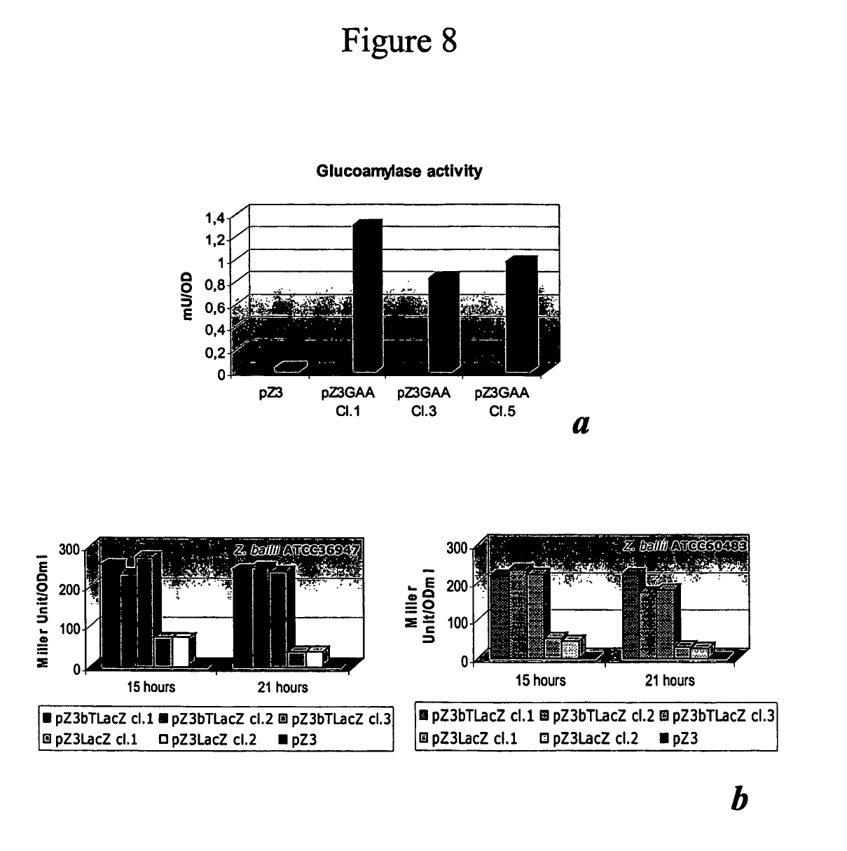

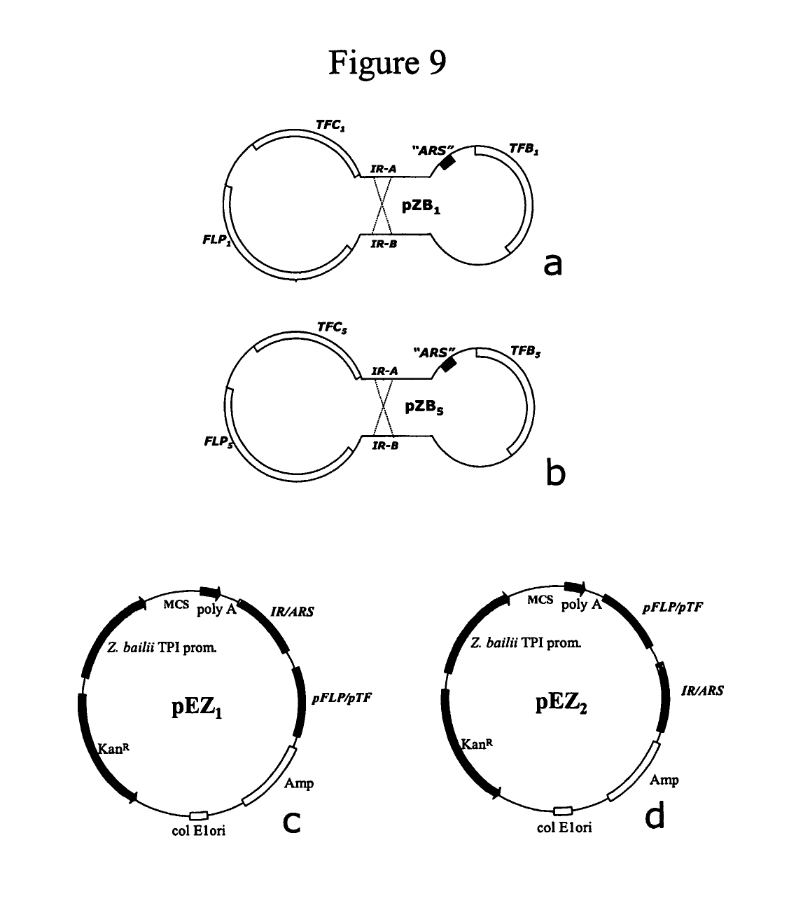

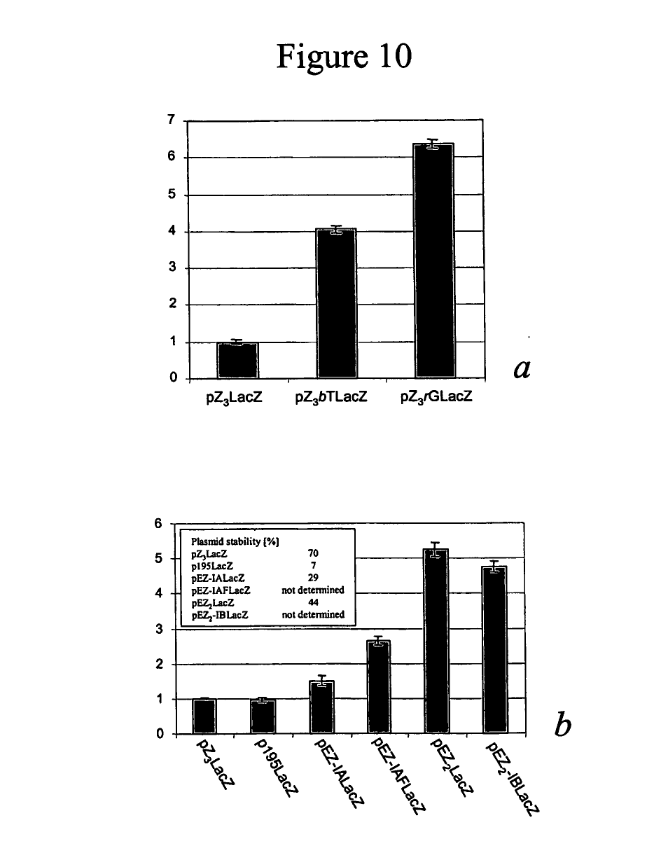

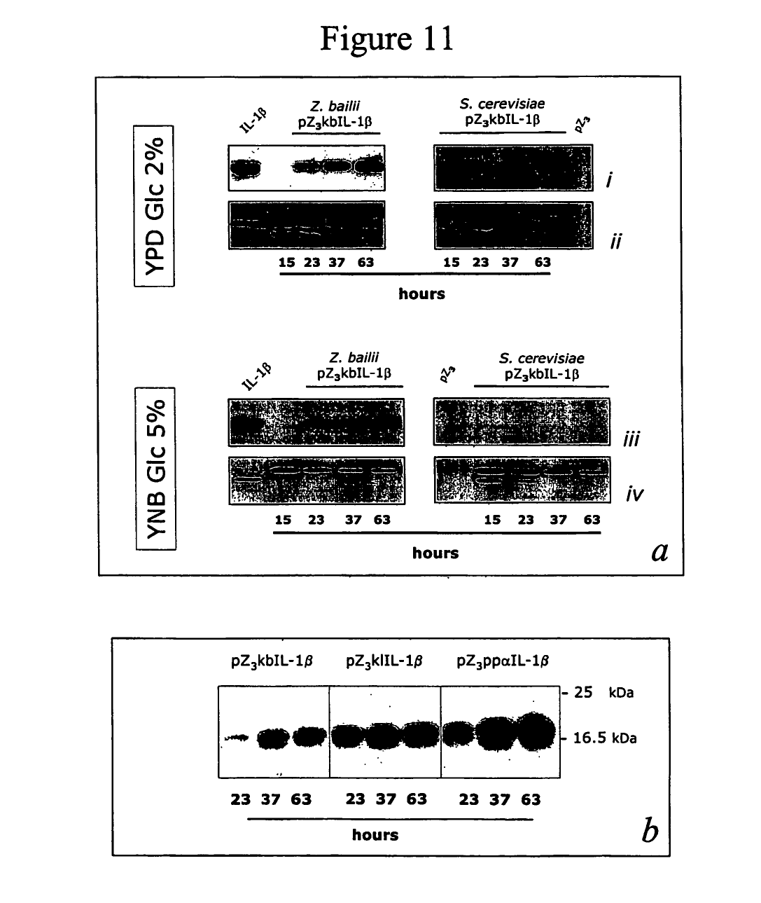

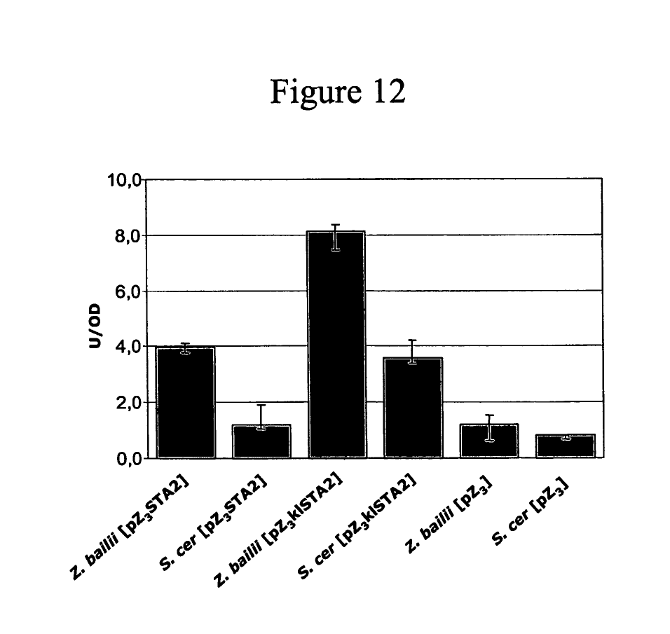

The Backbone of the new vector pZ₃ (FIG. 1a) is the basic S. cerevisiae expression plasmid YX022 (R&D Systems, Inc., Wiesbaden, D).

The ARS1-CEN4 fragment was taken from Ycplac33 (ATCC 87623, Genbank accession no.: X75456 L26352,). It was cutted ClaI-blunt/SpeI and cloned into pYX022 opened DraM-blunt/SpeI (in this way the plasmid lost completely the HIS gene).

The plasmid obtained was opened KpnI-blunt, and here the Kan cassette, derived from pFA6-KanMX4 (Wach et al., 1994 Yeast 10, 1793-1808) was inserted. The respective fragment was taken out cutting with SphI/SacI-blunt. This kanMX module contains the known kan^{r} open reading-frame of the E. coli transposon Tn903 fused to transcriptional and translational control sequences of the TEF gene of the filamentous fungus Ashbya gossypii (e.g. NRRL Y-1056). The described hybrid module permits efficient selection of transformants resistant against geneticin (G418).

The expression cassette based on the constitutive S. cerevisiae TPI promoter and the respective polyA, interspaced by the multi cloning site (MCS), as indicated in the Figure derives from the original pYX022 plasmid (see supplier's information). All the other plasmids indicated in the FIGS. 1 to 4 derive from pZ₃.

For the construction of the plasmid pZ₃kl (FIG. 1b), the signalling pre-sequence (16 aa) of the alpha-subunit of the K1 killer toxin of K. lactis (Stark M. J. et al., 1986, EMBO J. 5,1995-2002) was functionally linked to the TPI promoter of the pZ₃ plasmid, in order to lead the secretion of the protein of interest.

For the construction of the plasmid pZ₃ppa (FIG. 1c), the pre-pro-α-factor signal sequence was similarly utilised and functionally inserted. The sequence was taken from the plasmid pPICZαA (Invitrogen BV, The Netherlands)

For the construction of the plasmid pZ₃klIL-1β (FIG. 2a), the coding sequence for the protein already fused with the killer toxin K. lactis signal sequence was taken cutting XbaI/EcoRI-bluntended from the plasmid pCXJ-kanl (Fleer R, et al., 1991, Gene 107, 285-95) and sub-cloned into the plasmid pZ₃ EcoRI bluntended and de-phosphorylated.

For the construction of the plasmid pZ₃ppαGFP (FIG. 2c), the fragment containing the α-factor pre-pro leader sequence in frame with the GFP coding sequence was cutted HindIII bluntended/BamHI from the plasmid pPICAGFP1 and sub-cloned in the plasmid pZ₃ opened EcoRI bluntended/BamHI and de-phosphorylated. The plasmid pPICAGFP1 was constructed according to Passolunghi, S., et al. by introduction of a PCR amplified GFP sequence in frame into the plasmid pPICZαA (Invitrogen BV, The Netherlands). The PCR technique is known in the art. Exemplary reference is made to Gelfand, D. H., et al., PCR Protocols: A Guide to Methods and Applications, 1990, Academic Press and Dieffenbach, C.

W. et al., PCR Primer: A Laboratory Manual, Cold Spring Harbor Laboratory Press, 1995.

For the construction of the plasmid pZ₃ppαIL-1β (FIG. 2b), the IL-1β was PCR amplified from the plasmid pZ₃klIL-1β.

The oligos for the amplification are the following:

| | |
|---|---|
| Primer: DrdI-IL | (SEQ ID NO.: 80) |

| | |
|---|---|
| 5′ AAGAGACTCCAACGTCGGGCACCTGTA 3′ | Tm: 63° C. |

| | |
|---|---|
| Primer: IL C-term | (SEQ ID NO.: 81) |

| | |
|---|---|
| 5′ AGAGGATTAGGAAGACACAAATTGCATGGTGA 3′ | Tm: 61° C. |

The following program was used for the amplification:

| | | | | |
|---|---|---|---|---|
| 94° C. | 5 | min | | |
| 94° C. | 45 | s | | |
| 27° C. | 45 | s | {close oversize brace} | 10 cycles |
| 72° C. | 2 | min | | |
| 94° C. | 45 | s | | |
| 50° C. | 45 | s | {close oversize brace} | 20 cycles |
| 72° C. | 2 | min | | |
| 72° C. | 7 | min | | |

| | |
|---|---|
| 4° C. | ∞ |

In this way a DrdI cutting site for sub-cloning the coding sequence of the IL-1β protein in frame with the α-factor pre-pro leader sequence was introduced. The plasmid pZ₃ppαGFP was opened EcoRI bluntended/BamHI. The PCR fragment was cutted DrdI bluntended/BamHI. Combination resulted in the plasmid pZ₃ppαIL-1β.

In the plasmid pZ₃kbIL-1β, the coding sequence of the interleukin was functionally linked to the deduced pre-leader sequence of the Z. bailii killer toxin zygocin (Genebank accession no.: AF515592; Weiler F. et al., 2002, Mol Microbiol. 46, 1095-105.). Essentially oligonucleotides were synthesized corresponding to the deduced pre-leader sequence of the Z. bailii killer toxin zygocin (SEQ ID No.: 59) and cloned into the plasmid pZ₃. Subsequently, the IL-1β was PCR amplified as explicated before and cloned in-frame to the zygocin pre-sequence.

For the construction of the plasmid pZ₃GAA (FIG. 3a), the coding sequence of the A. adeninivorans α-glucoamylase was cut BamHI bluntended from the plasmid pTS32x-GAA (Bui D. M., et al., 1996, Appl. Microbiol. Biotechnol. 45, 102-6) and inserted in the plasmid pZ₃ opened EcoRI bluntended and de-phosphorylated. For the construction of the plasmid pZ₃STA2, the coding sequence of the S. cerevisiae var. diastaticus amylase (comprising its own leader sequence) was cut XbaI/AseI-blunt from the plasmid pMV35 (Vanoni M. et al., 1989, Biochim Biophys Acta. 1008, 168-76) and inserted in the plasmid pZ₃ opened EcoRI-blunt. For the construction of the plasmid pZ₃klSTA2, the coding sequence of the same amylase but functionally linked to the K. lactis killer toxin leader sequence was cut XhoI/AseI-blunt from the plasmid pMV57 (Venturini M. et al., 1997, Mol Microbiol. 23, 997-1007) and inserted in the plasmid pZ₃ opened EcoRI-blunt.

For the construction of the plasmid pZ₃LacZ (FIG. 3b), the coding sequence of the bacterial β-galactosidase was cutted HindIII bluntended/BamHI from the plasmid pSV-β-galactosidase (Promega, Inc.) and inserted into the plasmid pZ₃ opened EcoRI bluntended/BamHI and dephosphorylated.

In the plasmid pZ₃bT (FIG. 4a), the TPI promoter of S. cerevisiae was substituted with the endogenous TPI promoter from Z. bailii. The sequence was PCR amplified from the genomic DNA of the Z. bailii strain ISA 1307, and the primers were designed according to the literature (Merico A., et al., 2001, Yeast 18, 775-80). Extraction of genomic DNA was performed according to the protocol published by Hoffman, C. S., et al., 1987, Gene 57, 267-72).

The oligos for the amplification are the following:

| | |
|---|---|
| TPIprob5 | (SEQ ID NO.: 82) |

| | |
|---|---|
| 5′ ATCGTATTGCTTCCATTCTTCTTTTGTTA 3′ | Tm: 59.6° C. |

| | |
|---|---|
| TPIprob3 | (SEQ ID NO.: 83) |

| | |
|---|---|
| 5′ TTTGTTATTTGTTATACCGATGTAGTGTC 3′ | Tm: 59.6° C. |

The following program was used for the amplification:

| | | | | |
|---|---|---|---|---|
| 94° C. | 5 | min | | |
| 94° C. | 45 | s | | |
| 57° C. | 45 | s | {close oversize brace} | 25 cycles |
| 72° C. | 1 | min 30 s | | |
| 72° C. | 7 | min | | |

| | |
|---|---|
| 4° C. | ∞ |

The PCR fragment was sub-cloned into the vector pST-Blue1 (Novagen, Perfect Blunt cloning Kit cat. no. 70191-4), according to the included protocol. Therefrom, the promoter was cut SnaBI/SacI and sub-cloned into the pZ₃ opened AatII bluntended/SacI (so to remove the S. cerevisiae TPI promoter), obtaining the desired plasmid.

For the construction of the plasmid pZ₃bTLacZ (FIG. 4b), the coding sequence of the bacterial β-galactosidase was cutted HindIII/BamHI bluntended from the plasmid pSV-β-galactosidase (Promega, Inc.; Genebank accession no.: X65335) and inserted into the plasmid pZ₃bT opened NheI bluntended and de-phosphorylated.

In the plasmid pZ₃rG, the TPI promoter of S. cerevisiae was substituted with the GAPDH promoter from Z. rouxii. The sequence was PCR amplified from genomic DNA of the Z. rouxii. strain LST 1, and the primers were designed according to the literature (Ogawa Y. et al., 1990, Agric Biol Chem. 54, 2521-9). Extraction of genomic DNA was performed according to the protocol previously mentioned. (Another possible strain is Z. rouxii NRRL Y-229.)

The oligos for the amplification are the following:

| | |
|---|---|
| pZrGAPDH_fwd | (SEQ ID NO.: 93) |

| | |
|---|---|
| 5′ TGCAGAAAGCCCTAAGATGCT 3′ | Tm: 60.3° C. |

| | |
|---|---|
| pZrGAPDH_rev | (SEQ ID NO.: 94) |

| | |
|---|---|
| 5′ TGTCTGTGATGTACTTTTTATTTGATATG 3′ | Tm: 59.2° C. |

The following program was used for the amplification:

| | | | | |
|---|---|---|---|---|
| 94° C. | 5 | min | | |
| 94° C. | 15 | s | | |
| 57° C. | 30 | s | {close oversize brace} | 25 cycles |
| 72° C. | 45 | s | | |
| 72° C. | 7 | min | | |

| | |
|---|---|
| 4° C. | ∞ |

The obtained PCR fragment (708 bp) was sub-cloned into the vector pST-Blue1 (Novagen, Perfect Blunt cloning Kit cat. no. 70191-4), according to the included protocol. Therefrom, the promoter was cut SnaBI/SacI and sub-cloned into the pZ₃ opened AatII bluntended/SacI (so to remove the S. cerevisiae TPI promoter), obtaining the desired plasmid.

For the construction of the plasmid pZ₃rGLacZ (FIG. 4b), the coding sequence of the bacterial β-galactosidase was cut HindIII/BamHI bluntended from the plasmid pSV-β-galactosidase (Promega, Inc.; Genebank accession no.: X65335) and inserted into the plasmid pZ₃rG opened XhoI bluntended and de-phosphorylated.

DNA manipulation, transformation and cultivation of E. coli (DH5α), were performed following standard protocols (Sambrook J., et al., Molecular Cloning: A Laboratory Manual, 2nd edn., Cold Spring Harbor Laboratory, New York, 1989). Also other basic molecular biology protocols were performed following this manual if not otherwise stated. All the restriction and modification enzymes utilised are from NEB (New England Biolabs, UK) or from Roche Diagnostics.

### Example 2

### Transformation of Z. bailii

Transformations of all the Z. bailii and the S. cerevisiae (NRRL Y-30320) strains were performed basically according to the LiAc/PEG/ss-DNA protocol (Agatep, R., et al., 1998, Transformation of Saccharomyces cerevisiae by the lithium acetate/single-stranded carrier DNA/polyethylene glycol (LiAc/ss-DNA/PEG) protocol. Technical Tips Online (http://tto.trends.com)). After the transformation, Z. bailii cells were recovered with an incubation of 16 hours in YP medium, comprising 2% w/V of fructose as carbon source (YPF), and 1 M sorbitol, at 30° C. The cell suspension was then plated on selective YPF plates with 200 mg/l G418 (Gibco BRL, cat. 11811-031). Single clones appeared after 2-3 days at 30° C. From then on the transformants were grown either in rich or in minimal medium having glucose as carbon source and 200 mg/l G418 for maintenance of the selection. For S. cerevisiae cells, the procedure was the same, except for the carbon source, that remained glucose in all the steps, and for the G418 concentration, optimised for our strain to 500 mg/l.

### Example 3

### Expression and Secretion of Interleukin 1-β in Z. bailii

In order to check the secretory capability of the yeast Z. bailii and to compare it with the well known host S. cerevisiae, both yeasts were transformed (according to Example 2) with the plasmid pZ₃klIL-1β (FIG. 2a). Independent transformants were shake flask cultured in minimal medium (YNB, 1.34% w/V YNB from Difco Laboratories, Detroit, Mich. #919-15, 5% w/V Glucose, complemented with Histidine, Uracil and Leucine, FIG. 5a, left panel) or in rich medium (YPD, 5% w/V Glucose, 2% w/V Peptone, 1% w/V Yeast extract, FIG. 5a, right panel). FIG. 5a shows the cell density (OD 660 nm) and the glucose consumption during the kinetics of growth. The glucose consumption was determined using a commercially available enzymatic kit from Boehringer Mannheim GmbH, Germany (Cat #716251), according to the manufacturer's instructions. During the kinetics, samples were collected at the indicated times (see “hours” of FIG. 5b, c, d). Cells were harvested (a culture volume corresponding to 10⁸ cells) by centrifugation (10 min 10.000 rpm). 1 volume 2× Laemmli Buffer (Laemmli, U.K., 1970, Nature 227, 680-5) was added to the supernatants of said samples, they were boiled 3-5 minutes and stored at −20° C. until loading or loaded directly on a polyacrylamide gel.

The cell pellets of said samples were resuspended in 5 ml 20% TCA, centrifuged (10 min at 3000 rpm) and the resulting pellets were resuspended in 150 μl 5% TCA. Samples were subsequently centrifuged for 10 min at 3000 rpm, and the pellet was resuspended in Laemmli Buffer (100 μl). In order to neutralise the samples, 1 M Tris base was added (50 μl). After 3-5 min at 99° C. the samples are ready to be loaded on a polyacrylamide gel (alternatively, they can be stored at −20° C.).

Samples were loaded on standard polyacrylamide gels (SDS-PAGE, final concentration of the separating gel: 15%); after protein separation, gels were blotted (1 h, 250 mA) to nitrocellulose membranes (protran BA 85, Schleicher & Schuell). Immunodecoration: after 1 h (RT) of saturation in TBS 1× (1.2 g/l Tris base; 9 g/l NaCl)+5% NFM (non fat milk), 0.2% Tween-20, the membranes were incubated overnight at 4° C. with the primary antibody against interleukin (rabbit polyclonal antibody IL-1β(H-153) from Santa Cruz Biotechnology, Inc. cat. n^{o} sc-7884) diluted 1:200 in TBS 1× (1.2 g/l Tris base; 9 g/l NaCl)+5% NFM. After intensive and repeated washes in TBS+0.2% Tween-20, the secondary antibody (antirabbit IG horseradish peroxidase-conjugated, Amersham Biosciences, UK cat n^{o} NA934) was added (1:10.000 in TBS 1×+5% NFM) and left in incubation for 1 h (RT). The proteins were visualised using ECL Western Blotting System (Amersham Biosciences, UK) according to the manufacturer's instructions.

The data obtained by Western Blot performed on the supernatant highlight the surprisingly good secretory capability of Z. bailii cells (see FIG. 5c), both in minimal and in rich medium. Remarkably, the signal corresponding to the secreted protein is significantly more intense compared to the signal obtained from S. cerevisiae cells, in agreement with the lower signal revealed in Z. bailii crude cell extracts (FIG. 5b). Moreover, the difference in the secreted levels of proteins is even more pronounced in minimal medium respect than in rich medium (for a comparison: FIG. 5c, left and right panel). These conclusions can be done either considering samples loaded rectifying the OD (FIG. 5c) or either considering equal volumes of loaded samples (FIG. 5d).

Similarly, Z. bailii and S. cerevisiae cells were transformed with the plasmid pZ₃ppaIL-1β. In this case the same protein (interleukin) is functionally fused with the leader sequence of the S. cerevisiae α-factor pheromone. As previously described, cells were shake flask cultured in rich YPD or in minimal YNB medium, samples were collected and prepared for protein SDS-PAGE separation.

The Western Blot (FIG. 6a) once more revealed the surprisingly better secretion occurring in Z. bailii if compared to S. cerevisiae: the signals obtained from the crude extracts (i for YPD, iii for YNB medium) are more intense in the latter strain, suggesting that the product is shorter retained and therefore more efficiently secreted from Z. bailii cells. This observation is consistent with the fact that the signals corresponding to the product secreted into the medium are more intense in Z. bailii samples than in S. cerevisiae ones (ii for YPD, iv for YNB medium; in this case a positive signal is present only in Z. bailii samples).

Importantly, the process of expression, secretion and accumulation of heterologous proteins in the culture medium can be obtained not only by changing the leader sequence, but also by utilising the same leader sequence but changing the heterologous protein expressed. Z. bailii cells were transformed with the plasmid pZ₃ppαGFP, shake flask cultured in minimal YNB medium, samples were collected and prepared for protein SDS-PAGE separation. The Western Blot analyses performed as previously described, except for the primary antibody utilised (anti-GFP, Clontech, Inc.) and its concentration (1:500), show a band of the expected dimension that is present only in the supernatant of the Z. bailii cells expressing the GFP heterologous protein (FIG. 6b) and not in the control strain, transformed with the empty plasmid.

The data obtained underline the possibility to utilise Z. bailii as a host for the process to express different heterologous proteins and to secrete them, leading the secretion with heterologous leader sequences. Remarkably, the level of secreted proteins are higher compared with the levels obtained in S. cerevisiae, and the difference is even more pronounced, in chemically defined culture medium.

### Example 4

### Expression and Secretion of Interleukin 1-β in a Z. bailii Bioreactor Batch Cultivation with High Sugar Concentration.

Z. bailii cells transformed (according to Example 2) with the plasmid pZ₃klIL-1β (FIG. 2a) and previously analysed for interleukin 1-β production in shake flask culture (see Example 3), were batch cultivated in a 2 l laboratory bioreactor (fermentor, Biolafitte & Moritz, Mod. Prelude—France) in a chemically defined medium with high glucose content (27% w/V Glucose, 4% w/V (NH₄)₂SO₄, 0.4% w/V MgSO₄, 2.4% w/V KH₂PO₄, vitamins according to Verduyn, C., et al., 1992, Yeast 8, 501-17, wherein the final concentration of vitamins was set to be 24 times in respect to the indicated concentrations and trace elements according to Verduyn, C., et al., 1992, Yeast 8, 501-17, wherein the final concentration of trace elements was also set to be 24 times in respect to the indicated concentrations. (Depending on the salt tolerance of the production strain it might be useful in this context to add only a partial quantity of the salts with the glucose to the initial medium and to add the rest of the salts after the bioreaction (fermentation) has proceeded a sufficient amount of time.) The pH control (value: pH 5) is performed by the addition of 2M KOH. G418 was added to a concentration of 200 mg/l G418, antifoam was added as necessary). The inoculum was prepared by pre-growing the yeast in shake flask (with a headspace-to-culture volume ratio of 4) in YPD rich medium (see above), with the addition of 200 mg/l G418. Cells were harvested, washed with deionised water and inoculated in the final medium at OD 1.68 in the bioreactor. Cell culture was flushed with 90 l/h of air and the dissolved oxygen concentration was maintained at 40% of air saturation, varying the stirrer speed. FIG. 7a shows the growth kinetics (cell density, OD 660 nm), together with the glucose consumption, the ethanol production and the biomass produced (dry weight g/l). The glucose consumption and the ethanol production were determined by using commercial enzymatic kits (Boehringer Mannheim GmbH, Germany Kits Cat #716251 and 0176290, respectively), according to the manufacturer's instructions. The determination of the cellular dry weight (biomass) was performed as described before (Rodrigues, F. et al, 2001, Appl. Environ. Microbiol. 67, 2123-8). Samples were collected at the indicated times and prepared for protein SDS-PAGE separation. The Western Blot analysis (performed as described in Example 3) shows a very strong and clean signal accumulating during time corresponding to the secreted product (lanes 2 to 5), and confirms the minimal retention of heterologous protein produced within the cells (lanes 6 to 9, FIG. 7b). This example shows the surprising and advantageous characteristic of Z. bailii cells to be able to grow as well as express and secrete a heterologous protein even at very high sugar concentrations. Reportedly S. cerevisiae does not grow any more or can grow only very poorly at such high sugar concentrations (see for example Porro, D., et al., 1991, Res. Microbiol. 142, 535-9).

### Example 5

### Expression and Secretion of Glucoamylase in Z. bailii

Z. bailii cells were transformed (according to Example 2) with the plasmid pZ₃GAA (FIG. 3), and with the empty plasmid pZ₃, as a control. Independent transformants were shake flask cultured in minimal YNB medium with 2% w/V Glucose as a carbon source (+0.67 % w/V YNB and aa, according to the manufacturer's protocol) till mid-exp phase (also referred to as mid-log). The β-glucoamylase activity was determined as follows: after cell density determination, the cells were harvested in order to rescue the culture supernatant. 15 μl/ml 3M NaAc, pH 5.2 and 20 μl/ml 1% w/V Starch (Fluka 85642—high solubility—) were added. Subsequently, the samples were mixed well and incubated at the desired temperature (this experiment: 50° C.). At time zero and every following 20 min, 1 ml of the incubated medium is taken, ice-cooled for 2 min, 50 μl of Lugol solution (Fluka 62650) were added, shaken quickly and read at the spectrophotometer at λ580 nm. The slope of the resulting values corresponds to the glucoamylase activity. FIG. 8 shows the glucoamylase activity of three independent clones expressing the GAA and one negative control. The enzymatic activity is expressed in mU/OD, and it is calculated considering that 1U corresponds to the variation of 1 OD in 1 min. The values reported in the graphic were subtracted of the basic activity level of Z. bailii, as measured in the control sample.

Z. bailii and S. cerevisiae cells were transformed (according to Example 2) with the plasmids pZ₃STA2 and pZ₃klSTA2, and with the empty plasmid pZ₃, as a control. Independent transformants were shake flask cultured in minimal YNB medium with 2% w/V fructose as a carbon source (+0.67 % w/V YNB and aa, according to the manufacturer's protocol) till mid-exp phase (also referred to as mid-log). The α-glucoamylase activity was determined according to the literature (Modena et al., 1986, Arch of Biochem. And Biophys. 248, 138-50) as follows: after cell density determination, the cells were harvested in order to rescue the culture supernatant, and an aliquot of said supernatant is used for preparing the following reaction mix:

| | | |
|---|---|---|
| Supernatant | 100 | μl |
| Maltotriose 400 mM | 6.3 | μl |
| NaAc 200 mM pH 4.6 | 125 | μl |
| H₂O | 18.7 | μl |
| total | 250 | μl |

The mix is incubated for 1 hour at 37° C. under slow agitation, and after that time an aliquot of said mixture is used to evaluate the reaction. The product of maltotriose degradation is glucose, and its concentration can be determined using a commercially available enzymatic kit from Boehringer Mannheim GmbH, Germany (Cat #716251). 1U of glucoamylase specific activity is the quantity of enzyme necessary to release 1 μmol min−1 of glucose in said condition.

### Example 6

### Expression of β-galactosidase (β-gal) in Z. bailii

Z. bailii cells were transformed (according to Example 2) with the plasmid pZ₃LacZ (FIG. 3b), with the plasmid pZ₃bTLacZ (FIG. 4b), with the plasmid pZ3rGLacZ, and with the empty plasmid pZ₃, as a control. Independent transformants were shake flask cultured in YPD medium (see description above) with 2% w/V Glucose as a carbon source till mid-exp phase. β-galactosidase activity determination: after cell density determination, 1 ml culture is harvested into an eppendorf tube, spun for 5 minutes (to get a hard pellet), aspirated with a pipet, (not using the vacuum line!), washed in 1 ml Z buffer [w/o BME—betamercaptoethanol—; Z buffer: 16.1 g/l Na₂HPO₄.7H₂O, 5.5 g/l NaH₂PO₄.H₂O, 0.75 g/l KCl, 0.246 g/l MgSO₄.7H₂O], repelleted, suspended in 150 μl Z buffer (with BME, 27 μl/10 ml), 50 μl chloroform are added, 20 μl 0.1% SDS and vortexed vigorously for 15″. 700 μl of pre-warmed ONPG (o-nitrophenyl β-D-galactopyranoside, Sigma N-1127, 1 mg/ml in Z+BME) are added, and the reaction is started at 30° C. (20′ to 3 hr), checking the time. When the suspension turns yellow the reaction is stopped by addition of 0.5 ml of 1 M NaCO₃; after centrifugation for 10 min at maximum speed the sample is read at the spectrophotometer at λ420.

FIG. 8b shows the β-gal activity of three independent clones expressing the β-gal under control of the Z. bailii TPI promoter, two independent clones expressing the β-gal under control of the S. cerevisiae TPI promoter and one negative control (see the legend of the figure for indications of the respective clones). The enzymatic activity is expressed as Miller Unit/OD and it is calculated according to the following formula:

$$Miller Units = \frac{{A}_{420} \times 1000}{{A}_{660} \times time ( min ) \times Vol ( ml )}$$

As it is readily visible, the expression from the endogenous TPI promoter is much stronger (4-5 times) than from the respective promoter from S. cerevisiae.

A similar series of experiments was performed in order to evaluate the efficiency of the plasmids based on the sequences of the endogenous Z. bailii plasmid in improving the expression levels of heterologous proteins. Z. bailii cells were transformed (according to Example 2) with the following plasmids: pZ₃LacZ (FIG. 3b), p195LacZ, pEZ-IALacZ, pEZ-IAFLacZ, pEZ₂LacZ and pEZ₂-IBLacZ. Independent transformants were grown till mid-log phase and β-galactosidase activity measured, as previously described. The corresponding data are reported in FIG. 10b.

### Example 7

### Isolation of an Endogenous Z. bailii Plasmid

Z. bailii strains ATCC 36947 and NCYC 1427 were cultivated and their endogenous plasmid was extracted, resulting in the plasmids pZB₁ and pZB₅ (see FIGS. 9a and b). The protocol used was a modification of a protocol by Lorincz, A., 1985, BRL Focus 6, 11, and uses glass beads to break the cells. After the DNA extraction, samples were loaded on an agarose gel and the band corresponding to the plasmid was eluted (Qiagen, QIAquick Gel Extraction Kit cat n^{o} 28704). The plasmid extracted from NCYC 1427 was cut with EcoRI and some of the fragments were sequenced. These sequences correspond to SEQ ID No.: 63, SEQ ID No.: 64, SEQ ID No.: 65, SEQ ID No.: 66, SEQ ID No.: 67, SEQ ID No.: 68, SEQ ID No.: 69 or SEQ ID No.: 70, respectively.

### Example 8

### Sequence Amplification of the Open Reading Frames and of Structural Sequences of the Endogenous Z. bailii Plasmids

The genomic DNA extracted from the Z. bailii strains ATCC 36947 and NCYC 1427 were used as a template for the amplification of the open reading frames and of structural sequences of the endogenous Z. bailii plasmids.

The oligos for the amplification are the following:

| | |
|---|---|
| 5FLP | (SEQ ID NO.: 84) |

| | |
|---|---|
| 5′-TAGCTACTCTTCTCCAGGTGTCATTAG-3′ | Tm: 63.4 |

| | |
|---|---|
| 3FLP | (SEQ ID NO.: 85) |

| | |
|---|---|
| 5′-CCTATGTCCGAGTTTAGCGAGCTTG-3′ | Tm: 64.6 |

| | |
|---|---|
| 5TFC | (SEQ ID NO.: 86) |

| | |
|---|---|
| 5′-AGAATGAACTCAGAGTTGTCTCTTG-3′ | Tm: 59.7 |

| | |
|---|---|
| 3TFC | (SEQ ID NO.: 87) |

| | |
|---|---|
| 5′-ATTCTATTGGGTATGTCCCCTG-3′ | Tm: 58.4 |

| | |
|---|---|
| 5TFB | (SEQ ID NO.: 88) |

| | |
|---|---|
| 5′-GTTTTTAATTTTGAAGCTCACCTTTAATTG-3′ | Tm: 58.6 |

| | |
|---|---|
| 3TFB | (SEQ ID NO.: 89) |

| | |
|---|---|
| 5′-ATTATGTTCTCCAGGGAAGAGGTTAG-3′ | Tm: 61.6 |

| | |
|---|---|
| 5IRAARS | (SEQ ID NO.: 90) |

| | |
|---|---|
| 5′-AGAATCAATCATTTAGTGTGGCAGGAG-3′ | Tm: 61.9 |

| | |
|---|---|
| 3IRAARS | (SEQ ID NO.: 91) |

| | |
|---|---|
| 5′-TAAAAACTGCCCGCCATATTTCGTC-3′ | Tm: 61.3 |

The following program was used for the amplification:

| | | | | |
|---|---|---|---|---|
| 94° C. | 5 | min | | |
| 94° C. | 15 | s | | |
| 58° C. | 30 | s | {close oversize brace} | 25 cycles |
| 72° C. | 2 | min | | |
| 72° C. | 7 | min | | |

| | |
|---|---|
| 4° C. | ∞ |

The amplified fragments, sub-cloned into the vector pST-Blue1 (Novagen, Perfect Blunt cloning Kit cat. no. 70191-4), were sequenced and correspond to SEQ ID No.: 71 (IR-ARS), SEQ ID No.: 72 (FLP), SEQ ID No.: 74 (TFB) and SEQ ID No.: 76 (TFC), respectively.

These coding sequences are used for the construction of the expression plasmid pEZI, according to FIG. 9b.

### Example 9

### Construction of Expression Plasmids Based on Replication and Stability Sequences from the Z. bailii pSB2 Plasmid

The backbone of the new vectors is the basic S. cerevisiae multicopy plasmid Yeplac 195 (Gietz and Sugino, 1988, Gene 74, 527-34) modified to the expression plasmid pBR195, as described in Branduardi (2002, Yeast 19, 1165-70).

For the construction of the plasmid p195, the plasmid pBR195 was cut AatII/ApaI-blunt in order to excise the URA marker and the Kan^{R} cassette, excised SphI/SacI-blunt from pFA6-KanMX4 (Wach et al., 1994 Yeast 10, 1793-1808) was here inserted. From this plasmid derives the plasmid p195LacZ: the LacZ gene was sub-cloned from the plasmid pZ₃LacZ cut SphI/NheI into the new plasmid p195, opened with the same enzymes.

For the construction of the plasmids pEZ-IA and pEZ-IALacZ, the plasmids p195 and p195LacZ were opened NarI/StuI-blunt, in order to remove the S. cerevisiae 2 μm-ori. The PCR fragment corresponding to the IR-A and ARS sequence from the pSB2 (see previous example for amplification detail) was excised EcoRI-blunt from the pST-Blue1 plasmid and sub-cloned into the opened vectors just described.

For the construction of the plasmid pEZ-IAFLacZ, the plasmid pEZ-IALacZ was SmaI opened, and there the fragment corresponding to the FLP and the sequence containing its promoter, derived from the pST-Blue1 plasmid opened Acc1-blunt/SnaBI, was there sub-cloned. Said sequence was PCR amplified from the genomic DNA extracted from the Z. bailii strains ATCC 36947.

The oligos for the amplification are the following:

pFLP (SEQ ID NO.: 95)

| | |
|---|---|
| 5′-ACGCAAGAGAGAACTCTGAGTTCAT-3′ | Tm: 61.3 |

| | |
|---|---|
| 3FLP | (SEQ ID NO.: 85) |

| | |
|---|---|
| 5′-CCTATGTCCGAGTTTAGCGAGCTTG-3′ | Tm: 64.6 |

The following program was used for the amplification:

| | | | | |
|---|---|---|---|---|
| 94° C. | 5 | min | | |
| 94° C. | 15 | s | | |
| 58° C. | 30 | s | {close oversize brace} | 29 cycles |
| 72° C. | 1 | min 30 s | | |
| 72° C. | 7 | min | | |

| | |
|---|---|
| 4° C. | ∞ |

For the construction of the plasmids pEZ₂ and pEZ₂LacZ, the plasmids pEZ-IA and pEZ-IALacZ were opened SmaI and the PCR fragment corresponding to the sequences of FLP and TFC and the respective promoters was excided SnaBI/SalI-blunt from the pST-Blue1 plasmid and sub-cloned into the opened vectors just described.

The oligos for the amplification are the following:

| | |
|---|---|
| 5FLP | (SEQ ID NO.: 84) |

| | |
|---|---|
| 5′-TAGCTACTCTTCTCCAGGTGTCATTAG-3′ | Tm: 63.4 |

| | |
|---|---|
| 3TFC | (SEQ ID NO.: 87) |

| | |
|---|---|
| 5′-ATTCTATTGGGTATGTCCCCTG-3′ | Tm: 58.4 |

The following program was used for the amplification:

| | | | | |
|---|---|---|---|---|
| 94° C. | 5 | min | | |
| 94° C. | 15 | s | | |
| 58° C. | 30 | s | {close oversize brace} | 25 cycles |
| 72° C. | 1 | min 30 s | | |
| 72° C. | 7 | min | | |

| | |
|---|---|
| 4° C. | ∞ |

For resulting in the plasmid pEZ₂ an additional cloning step was required, in order to re-insert the polyA: the polyA was excised NaeI/NheI-blunt from the plasmid pYX022 and was sub-cloned in the transitory plasmid BamHI-blunt and de-phosphorylated.

For the construction of the plasmid pEZ₂-IBLacZ, the plasmid pEZ2LacZ was opened SalI-blunt and de-phosphorylated, and the fragment IR-B was therein sub-cloned. That fragment was EcoRI-blunt extracted from pST-Blue1 (see previous example).

### Example 10

### Plasmid Stability Determination

The stability of the plasmids described in the previous example was determined as follows: independent Z. bailii transformants bearing the different plasmids were inoculated at a cellular density of 5×10³ cells/ml in rich media (YPD) and in rich selective media (YPD+G418), respectively. At T₀ of the inoculum and then after 10 and 20 generations, 500 cells from any culture were plated 3 times on selective and non-selective agar plates, and subsequently incubated at 30° C. till the colonies became visible. The ratio between the mean of the colony number grown on selective medium and the mean of the colony number grown on non selective medium gives the percentage of mitotic stability.

1-21. (canceled)

22. A process for the production of a protein comprising the steps of:
a) culturing a Zygosaccharomyces bailii strain,
b) expressing and secreting a protein,
c) isolating the protein.

23. The process of claim 22, wherein the Z. bailii strain is transformed with a vector comprising a DNA sequence coding for the protein, functionally linked to a signal sequence leading to the secretion of the protein and further functionally linked to a promoter.

24. The process of claim 23, wherein the vector is an extra-chromosomal plasmid.

25. The process of claim 24, wherein the plasmid is derived from an endogenous episomal plasmid from a Z. bailii strain.

26. The process of claim 23, wherein the plasmid comprises sequences for replication, stabilization, or plasmid copy number control, obtainable from Z. bailii.

27. The process of claim 25, wherein the plasmid comprises at least 35 bases of one of the sequences selected from the group consisting of SEQ ID No.: 63, SEQ ID No.: 64, SEQ ID No.: 65, SEQ ID No.: 66, SEQ ID No.: 67, SEQ ID No.: 68, SEQ ID No.: 69, SEQ ID No.: 70, and SEQ ID No.: 71.

28. The process of claim 23, wherein the promoter is a triose-phosphate isomerase promoter, obtainable from Saccharomyces cerevisiae or from Z. bailii.

29. The process of claim 23, wherein the promoter is a glyceraldehyde phosphate dehydrogenase promoter, obtainable from Saccharomyces cerevisiae, Z. bailii or Z. rouxii.

30. The process of claim 23, wherein the signal sequence is a continuous stretch of 15 to 60 amino acids, comprising one or more positively charged amino acid(s) followed by a stretch of about 5 to 10 hydrophobic amino acids, which are optionally interrupted by non-hydrophobic residues.

31. The process of claim 23, wherein the signal sequence is selected from the list consisting of SEQ ID NO.: 1, SEQ ID NO.: 3, SEQ ID NO.: 5, SEQ ID NO.: 7, SEQ ID NO.: 9, SEQ ID NO.: 11, SEQ ID NO.: 13,SEQ ID NO.: 15,SEQ ID NO.: 17, SEQ ID NO.: 19, SEQ ID NO.: 21, SEQ ID NO.: 23, SEQ ID NO.: 25, SEQ ID NO.: 27, SEQ ID NO.: 29, SEQ ID NO.: 31, SEQ ID NO.: 33, SEQ ID NO.: 35, SEQ ID NO.: 37, SEQ ID NO.: 39, SEQ ID NO.: 41, SEQ ID NO.: 43, SEQ ID NO.: 45, SEQ ID NO.: 47, SEQ ID NO.: 49, SEQ ID NO.: 51, SEQ ID NO.: 53, SEQ ID NO.: 55, SEQ ID NO.: 57, SEQ ID NO.: 59, and SEQ ID NO.: 61.

32. The process of claim 22, wherein the Z. bailii strain is transformed with a vector comprising the DNA sequence coding for the protein, functionally linked to the signalling pre-sequence of the alpha-subunit of the K1 killer toxin of Kluyveromyces lactis and further functionally linked to the triose-phosphate isomerase promoter from S. cerevisiae.

33. The process of claim 32, wherein the vector is the plasmid pZ₃kl as shown in FIG. 1b.

34. The process of claim 22, wherein the Z. bailii strain is transformed with a vector comprising the DNA sequence coding for the protein, functionally linked to the signal sequence of the pre-pro α-factor of S. cerevisiae and further functionally linked to the triose-phosphate isomerase promoter from S. cerevisiae.

35. The process of claim 34, wherein the vector is the plasmid pZ₃ppα as shown in FIG. 1c.

36. The process of claim 23, wherein the DNA sequence coding for the protein is derived from animal, bacterial, fungal, plant, or viral sources.

37. The process of claim 23, wherein the Z. bailii strain that is transformed is selected from the list of: ATCC 36947, ATCC 60483, NCYC 1427 or ATCC 8766.

38. The process of claim 22, wherein the Z. bailii strain has been subjected to a selection process for improved secretion.

39. The process of claim 22, wherein the Z. bailii strain is cultivated in a chemically defined medium.

40. The process of claim 22, wherein the protein is isolated from the culture medium.

41. A Z. bailii strain, expressing and secreting a heterologous protein.

42. The Z. bailii strain of claim 41, wherein the cells are transformed with a vector comprising a DNA sequence coding for the heterologous protein, functionally linked to a signal sequence leading to the secretion of the protein and further functionally linked to a promoter.


SEQUENCE LISTING (95 sequences)

SEQ ID NO: 1 - DNA, 57 bp, Saccharomyces cerevisiae
cgacagtaaattttgccgaatttaatagcttctactgaaaaacagtggaccatgtga

SEQ ID NO: 2 - protein, 19 aa, Saccharomyces cerevisiae
MRFPSIFTAVLFAASSALA

SEQ ID NO: 3 - DNA, 255 bp, Saccharomyces cerevisiae
cgacagtaaattttgccgaatttaatagcttctactgaaaaacagtggaccatgtgaaaagatgcatctcatttatcaaacacataatattcaagtgagccttacttcaattgtattgaagtgcaagaaaaccaaaaagcaacaacaggttttggataagtacatatataagagggccttttgttcccatcaaaaatgttactgttcttacgattcatttacgattcaagaatagttcaaacaagaagattacaa

SEQ ID NO: 4 - protein, 85 aa, Saccharomyces cerevisiae
MRFPSIFTAVLFAASSALAAPVNTTTEDETAQIPAEAVIGYLDLEGDFDVAVLPFSNSTNNGLLFINTTIASIAAKEEGVSLDKR

SEQ ID NO: 5 - DNA, 63 bp, Aspergillus niger
atgatggtcgcgtggtggtctctatttctgtacggccttcaggtcgcggcacctgctttggct

SEQ ID NO: 6 - protein, 21 aa, Aspergillus niger
MMVAWWSLFLYGLQVAAPALA

SEQ ID NO: 7 - DNA, 93 bp, Bacillus sp.
atgcaaaacacagcgaaaaactccatctggcagagggtgcgccacagcgccattgccttatccgctctcagtttatcctttggcctgcaggcc

SEQ ID NO: 8 - protein, 31 aa, Bacillus sp.
MQNTAKNSIWQRVRHSAIALSALSLSFGLQA

SEQ ID NO: 9 - DNA, 63 bp, Aspergillus oryzae
atgcagtccatcaagcgtaccttgctcctcctcggagctatccttcccgcggtcctcggtgcc

SEQ ID NO: 10 - protein, 21 aa, Aspergillus oryzae
MQSIKRTLLLLGAILPAVLGA

SEQ ID NO: 11 - DNA, 75 bp, Bacillus amyloliquefaciens
atgaaacgagtgttgctaattcttgtcaccggattgtttatgagtttgtgtgggatcacttctagtgtttcggct

SEQ ID NO: 12 - protein, 25 aa, Bacillus amyloliquefaciens
MKRVLLILVTGLFMSLCGITSSVSA

SEQ ID NO: 13 - DNA, 51 bp, Saccharomycopsis fibuligera
atgttgatgatagtacagcttttggtctttgcactaggccttgctgttgct

SEQ ID NO: 14 - protein, 17 aa, Saccharomycopsis fibuligera
MLMIVQLLVFALGLAVA

SEQ ID NO: 15 - DNA, 51 bp, Saccharomycopsis fibuligera
atgttgttgattttggaactcttagtacttattatagggcttggagttgct

SEQ ID NO: 16 - protein, 17 aa, Saccharomycopsis fibuligera
MLLILELLVLIIGLGVA

SEQ ID NO: 17 - DNA, 66 bp, Hypocrea pecorina
atggcgccctcagttacactgccgttgaccacggccatcctggccattgcccggctcgtcgccgcc

SEQ ID NO: 18 - protein, 22 aa, Hypocrea pecorina
MAPSVTLPLTTAILAIARLVAA

SEQ ID NO: 19 - DNA, 63 bp, Hypocrea pecorina
atgaacaagtccgtggctccattgctgcttgcagcgtccatactatatggcggcgccgtcgca

SEQ ID NO: 20 - protein, 21 aa, Hypocrea pecorina
MNKSVAPLLLAASILYGGAVA

SEQ ID NO: 21 - DNA, 48 bp, Arxula adeninivorans
atgcgtcagtttctagcacttgctgctgctgcttccatagcggtggca

SEQ ID NO: 22 - protein, 18 aa, Arxula adeninivorans
MRQFLALAAAASIAVADS

SEQ ID NO: 23 - DNA, 57 bp, Homo sapiens
atgcagcgactatgtgtgtatgtgctgatctttgcactggctctggccgccttctct

SEQ ID NO: 24 - protein, 19 aa, Homo sapiens
MQRLCVYVLIFALALAAFS

SEQ ID NO: 25 - DNA, 75 bp, Rhizopus oryzae
atgcaactgttcaatttgccattgaaagtttcattctttctcgtcctctcttacttttctttgctcgtttctgct

SEQ ID NO: 26 - protein, 25 aa, Rhizopus oryzae
MQLFNLPLKVSFFLVLSYFSLLVSA

SEQ ID NO: 27 - DNA, 48 bp, Aspergillus niger
atgcagactctccttgtgagctcgcttgtggtctccctcgctgcggcc

SEQ ID NO: 28 - protein, 16 aa, Aspergillus niger
MQTLLVSSLVVSLAAA

SEQ ID NO: 29 - DNA, 54 bp, Homo sapiens
atgaagtgggtaacctttatttcccttctttttctctttagctcggcttattcc

SEQ ID NO: 30 - protein, 18 aa, Homo sapiens
MKWVTFISLLFLFSSAYS

SEQ ID NO: 31 - DNA, 78 bp, Saccharomyces cerevisiae
atgacgaagccaacccaagtattagttagatccgtcagtatattatttttcatcacattactacacctagtcgtagcg

SEQ ID NO: 32 - protein, 26 aa, Saccharomyces cerevisiae
MTKPTQVLVRSVSILFFITLLHLVVA

SEQ ID NO: 33 - DNA, 63 bp, Saccharomyces cerevisiae
atgggccacttagcgatccttttcagtattatcgctgtattgaatatagctacagctgttgca

SEQ ID NO: 34 - protein, 21 aa, Saccharomyces cerevisiae
MGHLAILFSIIAVLNIATAVA

SEQ ID NO: 35 - DNA, 48 bp, Kluyveromyces lactis
ataaaatgaatatattttacatatttttgtttttgctgtcattcgttc

SEQ ID NO: 36 - protein, 17 aa, Kluyveromyces lactis
MNIFYIFLFLLSFVQGL

SEQ ID NO: 37 - DNA, 69 bp, Kluyveromyces lactis
ataaaatgaatatattttacatatttttgtttttgctgtcattcgttcaaggtttggagcatactcatc

SEQ ID NO: 38 - protein, 23 aa, Kluyveromyces lactis
MKIYHIFSVCYLITLCAAAATTA

SEQ ID NO: 39 - DNA, 54 bp, Saccharomyces cerevisiae
atgtttgctttctactttctcaccgcatgcatcagtttgaagggcgtttttggg

SEQ ID NO: 40 - protein, 18 aa, Saccharomyces cerevisiae
MFAFYFLTACISLKGVFG

SEQ ID NO: 41 - DNA, 54 bp, Saccharomyces cerevisiae
atgtttaagtctgttgtttattcggttctagccgctgctttagttaatgcaggt

SEQ ID NO: 42 - protein, 18 aa, Saccharomyces cerevisiae
MFKSVVYSVLAAALVNAG

SEQ ID NO: 43 - DNA, 51 bp, Saccharomyces cerevisiae
atgtttaaatctgttgtttattcaattttagccgcttctttggccaatgca

SEQ ID NO: 44 - protein, 17 aa, Saccharomyces cerevisiae
MFKSVVYSILAASLANA

SEQ ID NO: 45 - DNA, 48 bp, Kluyveromyces lactis
atgctatctattctgttgagtttattatcattatcagggacccatgcg

SEQ ID NO: 46 - protein, 16 aa, Kluyveromyces lactis
MLSILLSLLSLSGTHA

SEQ ID NO: 47 - DNA, 48 bp, Kluyveromyces lactis
atgctatctattctgttgggtttattatcactatcagggacccatgcg

SEQ ID NO: 48 - protein, 16 aa, Kluyveromyces lactis
MLSILLGLLSLSGTHA

SEQ ID NO: 49 - DNA, 54 bp, Aspergillus niger
atgggcgtctctgctgttctacttcctttgtatctcctgtctggagtcacctcc

SEQ ID NO: 50 - protein, 18 aa, Aspergillus niger
MGVSAVLLPLYLLSGVTS

SEQ ID NO: 51 - DNA, 57 bp, Saccharomyces cerevisiae
atgcttttgcaagctttccttttccttttggctggttttgcagccaaaatatctgca

SEQ ID NO: 52 - protein, 20 aa, Saccharomyces cerevisiae
PMLLQAFLFLLAGFAAKISA

SEQ ID NO: 53 - DNA, 63 bp, Saccharomyces cerevisiae
atgcaaagaccatttctactcgcttatttggtcctttcgcttctatttaactcggctttgggt

SEQ ID NO: 54 - protein, 21 aa, Saccharomyces cerevisiae
MQRPFLLAYLVLSLLFNSALG

SEQ ID NO: 55 - DNA, 63 bp, Saccharomyces cerevisiae
atgcaaagaccatttctactcgcttatttggtcctttcgcttctatttaactcagctttgggt

SEQ ID NO: 56 - protein, 21 aa, Saccharomyces cerevisiae
MQRPFLLAYLVLSLLFNSALG

SEQ ID NO: 57 - DNA, 96 bp, Saccharomyces cerevisiae
atggtaggcctcaaaaatccatatacgcacactatgcaaagaccatttctactcgcttatttggtcctttcgcttctatttaactcagctttgggt

SEQ ID NO: 58 - protein, 32 aa, Saccharomyces cerevisiae
MVGLKNPYTHTMQRPFLLAYLVLSLLFNSALG

SEQ ID NO: 59 - DNA, 63 bp, Zygosaccharomyces bailii
atgaaagcagcccaaatattaacagcaagtatagtaagcttattgccaatatatactagtgct

SEQ ID NO: 60 - protein, 21 aa, Zygosaccharomyces bailii
MKAAQILTASIVSLLPIYTSA

SEQ ID NO: 61 - DNA, 415 bp, Zygosaccharomyces bailii
atgaaagcagcccaaatattaacagcaagtatagtaagcttattgccaatatatactagtgctagaaacatattagacagagaatacacagcaaacgaattaaaaactgcttttggagatgaagaaatttttacagatttgacgtatcacattcacgttaacgtcagtggcgaaattgactcttactatcataatttagtcaattttgtcgataacgctctagcaaacaaagatattaatagatatatatacgctatatttacacagcagacaaactatacagaggatgggctcattgagtacttaaatcattacgattcagagacttgcaaagatatcattactcagtataatgttaacgtagacactagtaactgtataagcaatactacagatcaagctagactccaacgtc

SEQ ID NO: 62 - protein, 139 aa, Zygosaccharomyces bailii
MKAAQILTASIVSLLPIYTSARNILDREYTANELKTAFGDEEIFTDLTYHIHVNVSGEIDSYYHNLVNFVDNALANKDINRYIYAIFTQQTNYTEDGLIEYLNHYDSETCKDIITQYNVNVDTSNCISNTTDQARLQRR

SEQ ID NO: 63 - DNA, 587 bp, Zygosaccharomyces bailii
ttatagatctaaaaataaataatataaattacctcatccagagtccccccccaaatccttattctaaataataagctactcctccccccccaggagtatttttagggggaggggggaccttaactcaagggggagtagttttgaggatcacatgggaagtatttaaataaatagtagttcttttgttttaaaaaggcctctccaaaagtaatacttttagggtaattactaagtataatatatattataagtaatagcctttatagcttaatggtaaagcagtaaattgaagatttacctatatgtagttcgattctcattaagggcaatataaataagctttttaatgggccaatagctgaaataagtaatattattgtaaatattgagacttgaactcaaatcttatgcacctaaaaacatatattttaaccaattaaattatatttactttattatttacttatataacttctactaattgtaaagtataaccagcttttttgttaacaacaaaaaccgagagggttcatgttatatataatttataattgttcttactttatttataaaagaataaccgaatg

SEQ ID NO: 64 - DNA, 435 bp, Zygosaccharomyces bailii
ttaatttaatttgtcgctaaataataatgttttaaatattataaatatttcaaccaaccaccccccccaaaagggggtggttggtggttggtcgtcaccaaccacctttggtgggtggtgccccctatgagttttcatattataaatataaaaacttttatggagggacctataagaaataattgaggaataattaataataagttgccctccttttttttctcttctccccaccctaaaaatactcctgggggggggagggagagaatgtatgtagtggggagggtgtaagttaataatagacttaaatagagttatataaaataacataaatatgcttaaaaataataataataatattaacagatagaagccaaagggtcaggcgctttctttgggagaaagagttagttagttcgaatctatcctatctga

SEQ ID NO: 65 - DNA, 299 bp, Zygosaccharomyces bailii
ttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataa

SEQ ID NO: 66 - DNA, 153 bp, Zygosaccharomyces bailii
ctccactgtaacatttcccactgtccttttcccatctttcattttacaatgagcaagtttcagaaaaaaaaatacaaatgggataagtgcaaaacattccatgtatctgtagcttccaatgttattcctctctccagagtcaggcttctgtgt

SEQ ID NO: 67 - DNA, 231 bp, Zygosaccharomyces bailii
aattcaatagatgatgatttaacttcatttaatgagaagatttcattagattcaacaaaatctggagattttgcataaacaactgatttattattagctttattttctaatccattaactaattgatcatacataatatagatgaataagaataatgaaactagtgcaataattgatccaattgatgctacataatttcaaccagcaaaggcatcagggtagtcaggaatt

SEQ ID NO: 68 - DNA, 52 bp, Zygosaccharomyces bailii
ctcgtaaaaacgagcatgagctgcgtcaggtcagccgtggatatcgttgcgg

SEQ ID NO: 69 - DNA, 116 bp, Zygosaccharomyces bailii
ctatctgcacgtgccaccggaggtgctgtgggagcgactgcggcacgatcgccatcggccgctgctgcaggtgccgcagccgaggcagcgcattttcgaactctacgcccagcgcg

SEQ ID NO: 70 - DNA, 268 bp, Zygosaccharomyces bailii
ccggccccttgttgcgggcggtgccgatcaggcgcagcgagttgatctcgcgcagccggtcctgggcataattgagcaaatcgtactcgtgcgcggcgatgcgctccttgccgatcgaattgacgtaatcgatcgcggcgccgagcccgatcgcctcgacgatcggcggcgtgccggcctcgaacttgtgcggcgggtcgccataggtgacccagtccttggcaacttcacggatcatttcgccgccgccgttgaacggccgcatcgc

SEQ ID NO: 71 - DNA, 869 bp, Zygosaccharomyces bailii
cacacagaaacagctatgaccatgatacgccaagcttaatacgactcactataggaaagctcggtaccacgcatgctgcagacgcgttacgtatcggatccagaattcgtgatattctattgggtatgtcccctgattcgacggcgtaaattgcgtgaatcttgtgttggcgctaatgaccgctttttggaattatgtgctatgcctctgccattggtatcaacagctgaaatatttgttgaagatcgaatatcttctattgtttctgagggtatccccgaagctatggcgaaagaaaggatctcttctcgtacttggatcggtacgagaagcaatagacgcacaatgcattgacgcatcttgttgataccgggtaatgtgagtcttctgggttctgttattgagtttaatatgtcgtccacctctgttctcgtatccattttgcgagtagcccgccatacagcacgtccaatacaggagaggccatttagcttcaggtgcagagaagacacagcatggtgctcaccttcgagtgtctcaatagatgattgagttgactgggcttccgtgaaagggcctttcgagagatcttcagaaataaaccagggttgcgcttcattagtaggtgttcctggaggactattgtcgctatctgctggactactgctaccaagtagtgaagggggtattctaaggctttcactctgttctgacactattataacattgccaaggccaatttgaaaggtttcgcgtatatgagtaaagagctcggtgcccttccagttggaatcaagccgttcaagcagatcgagagcataatcagagtccacatttccgcacgcaagagagaactctgagttcattct

SEQ ID NO: 72 - DNA, 1425 bp, Zygosaccharomyces bailii
  CDS (1)...(1425)
atgtccgagtttagcgagcttgtcagaattctcccattagaccaggttgcagaaataaagcgtattttgagtcgcggcgaccctatacctttacaaaggttagcttctctactaactatggtgatcctaacggtcaacatgtcaaaaaagaggaagagctctccaatcaagcttagcacctttactaaatatcgtagaaatgttgcgaagtcattgtattatgatatgtcaagcaagacagtattcttcgaataccatctcaaaaatacacaagatctacaggagggcctcgagcaagccattgcgccctacaatttcgtggtaaaggtgcacaagaagccaattgattggcagaaacagctctcaagcgtgcatgagaggaaagcgggccacagaagcattctcagcaacaatgttggcgccgagatctctaaactggctgagacgaaagattctacttggagttttatcgagagaacaatggatctgatagaagcccgcacccgccagcccacgacaagagttgcgtataggtttctgcttcaactcacattcatgaactgctgtagggctaatgatttgaaaaacgccgaccccagcacttttcaaatcatcgcagatcctcaccttggtcgtatattgcgggcctttgttccagagacaaagactagcattgaaaggtttatctattttttcccatgtaagggacgatgcgatccgcttttggctctagattcctatctcctgtgggttggcccagtgcccaaaactcagactaccgatgaagagactcaatatgattaccagcttcttcaagatactctcttgatttcgtacgacaggtttatcgccaaagaatcaaaggaaaatattttcaaaatacctaatgggcccaaagctcatttggggcggcatctaatggcatcataccttggaaacaacagtctcaagagcgaggccacactctacggcaactggtctgtggaaaggcaagagggcgtcagcaaaatggctgacagccgatacatgcacacggttaaaaaaagtccaccttcatatctatttgcatttttatccggctactacaaaaagtccaaccaaggcgagtacgtgctggctgaaacactgtataatcccctggattacgacaaaacacttccaataacaacgaacgagaaattgatctgtcggcggtacgggaaaaatgcgaaagtgataccaaaagacgcactgctgtatctctacacgtatgcgcagcagaagcgaaaacaattggccgatcccaatgagcaaaataggctattcagtagtgaatcaccagcgcatcccttcttaactcctcaatcgacaggctcatcgacacccttgacctggactgctccaaagacactctccactggtctaatgacacctggagaagagtag

SEQ ID NO: 73 - protein, 474 aa, Zygosaccharomyces bailii
MSEFSELVRILPLDQVAEIKRILSRGDPIPLQRLASLLTMVILTVNMSKKRKSSPIKLSTFTKYRRNVAKSLYYDMSSKTVFFEYHLKNTQDLQEGLEQAIAPYNFVVKVHKKPIDWQKQLSSVHERKAGHRSILSNNVGAEISKLAETKDSTWSFIERTMDLIEARTRQPTTRVAYRFLLQLTFMNCCRANDLKNADPSTFQIIADPHLGRILRAFVPETKTSIERFIYFFPCKGRCDPLLALDSYLLWVGPVPKTQTTDEETQYDYQLLQDTLLISYDRFIAKESKENIFKIPNGPKAHLGRHLMASYLGNNSLKSEATLYGNWSVERQEGVSKMADSRYMHTVKKSPPSYLFAFLSGYYKKSNQGEYVLAETLYNPLDYDKTLPITTNEKLICRRYGKNAKVIPKDALLYLYTYAQQKRKQLADPNEQNRLFSSESPAHPFLTPQSTGSSTPLTWTAPKTLSTGLMTPGEE

SEQ ID NO: 74 - DNA, 1075 bp, Zygosaccharomyces bailii
  CDS (1)...(1074)
atgttctccagggaagaggttagggcctccaggcccactaaagagatgaagatgatctttgatgtgcttatgacatttccttacttcgcggtacatgttccttccaagaatatacttatcacaccaaaaggcacagttgagatacctgaaaactatcaaaattatcccatattggccatcttctacgtcaaatatttaatgaagaaaaatccgtacgatcttcttccaagcaccgtgaactggccggaaccctatgtagtggtgaataccatcactaagcgtttccaggaccataaactatttgcaaacaaaaatgctgatgtctacgttgaaagacttcaaaatgcaattgcctcgggtattaagattcctgagtctaagaagaatgaacgattagggcagccaaaaaagacgaaaaatgttacaaaagagaattgaggagacctttattgatgccactaatgcgagaaaagaattggatgagtacttcagaaaacttcaggatggtacattaaccggagatttggagggtggcttgtgcaaggtcaaaacgctcatatcgtgtaaagctttgttcggaggacacacccaagaactccagtttatggccaccaatgttcgtaaagtctggataggggagatagtgtgcggcatggtttccaataaaaatgcaattgacgataatgatctcgaggaagaagagcgtaatgcatcgggcgaacaaactacgacagcccgagaggaatcagaggctctggataccacatccaatggtttggacgctctgaatactcaaattaatgccatagaaacggaggaatcattttgggaagctatcagggcgctccataatgagctacgcacctctccaacacagttagaagagtgcaggaaagcggcagtttttttactgggccataaaaaaatactccaaacatttacaaagcaaaaggatactgcccgcgctcttttttatataaatctcaaagagtgtctgggaaccagctggaatttagaatatacagaggcatcagatgcaagaaaaatggcaattaaaggtgagcttcaaaattaa

SEQ ID NO: 75 - protein, 341 aa, Zygosaccharomyces bailii
MFSREEVRASRPTKEMKMIFDVLMTFPYFAVHVPSKNILITPKGTVEIPENYQNYPILAIFYVKYLMKKNPYDLLPSTVNWPEPYVVVNTITKRFQDHKLFANKNADVYVERLQNAIASGIKIPESKKNERLGQPKKTKNVTKENGDLYCHCEKRIGVLQKTSGWYINRRFGGWLVQGQNAHIVSFVRRTHPRTPVYGHQCSSLDRGDSVRHGFQKCNRSRGRRACIGRTNYDSPRGIRGSGYHIQWFGRSEYSNCHRNGGIILGSYQGAPATHLSNTVRRVQESGSFFTGPKNTPNIYKAKGYCPRSFLYKSQRVSGNQLEFRIYRGIRCKKNGNRASKL

SEQ ID NO: 76 - DNA, 750 bp, Zygosaccharomyces bailii
  CDS (1)...(750)
atgaactcagagttctctcttgcgtacggaaatgtggactctgattatgctctcgatctgcttgaacggcttgattccaactggaagggcaccgagctctttactcatatacgcgaaacctttcaaattggccttggcaatgttatcatagtgtcagaacagagtgaaagccttagaatacccccttcactacttggtagcagtagtccagcagatagcgacaatagtcctccaggaacacctactaatgaagcgcaaccctggtttatttctgaagatctctcgaaaggccctttcacggaagcccagtcaactcaatcatctattgagacactcgaaggtgagcaccatgctgtgtcttctctgcacctgaagctaaatggcctctcctgtattggacgtgctgtatggcgggctactcgcaaaatggatacgagaacagaggtggacgacatattaaactcaataacagaacccagaagactcacattacccggtatcaacaagatgcgtcaatgcattgtgcgtctattgcttctcgtaccgatccaagtacgagaagagatcctttctttcgccatagcttcggggataccctcagaaacaatagaagatattcgatcttcaacaaatatttcagctgttgataccaatggcagaggcatagcacataattccaaaaagcggtcattagcgccaacacaagattcacgcaatttacgccgtcgaatcaggggacatacccaatag

SEQ ID NO: 77 - protein, 249 aa, Zygosaccharomyces bailii
MNSEFSLAYGNVDSDYALDLLERLDSNWKGTELFTHIRETFQIGLGNVIIVSEQSESLRIPPSLLGSSSPADSDNSPPGTPTNEAQPWFISEDLSKGPFTEAQSTQSSIETLEGEHHAVSSLHLKLNGLSCIGRAVWRATRKMDTRTEVDDILNSITEPRRLTLPGINKMRQCIVRLLLLVPIQVREEILSFAIASGIPSETIEDIRSSTNISAVDTNGRGIAHNSKKRSLAPTQDSRNLRRRIRGHTQ

SEQ ID NO: 78 - DNA, 453 bp, Saccharomyces cerevisiae
  promoter (1)...(450)
  misc_feature (451)...(453): start codon
ctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatg

SEQ ID NO: 79 - DNA, 499 bp, Zygosaccharomyces bailii
  promoter (1)...(496)
  misc_feature (497)...(499): start codon
ggatcgtattgcttccattcttcttttgttattcggcgcgattcgaattcatgacatcttttaaccgtccgcactacattactggctcaagaaaggattgataaatactaccaaggaacacgtgtatccatttgatactgtgctggttacaagacacatgctttacaagcacacttctatctctctcgactgaggcgaaacgtcgagtggtttgatatcaaatgcatgcgtgatatgcaccattatttttcccttttacttccgtcacgccggggctccacttttttgggttccacttttcttacgaccctcgacatccactaaacgaacaggaagtcaaagaacccctcgagtcacacggtgcgtatgcgctgttaacatatataaaggtcacctttccctgctcaaaagagtcttagcaggctgttaacttcactctctatcgatccatagaatctaactaacaagagactacatcggtataacaaataacaaaatg

SEQ ID NO: 80 - DNA, 27 bp, Artificial Sequence
  PCR Primer
aagagactccaacgtcgcgcacctgta

SEQ ID NO: 81 - DNA, 32 bp, Artificial Sequence
  PCR Primer
agaggattaggaagacacaaattgcatggtga

SEQ ID NO: 82 - DNA, 29 bp, Artificial Sequence
  PCR Primer
atcgtattgcttccattcttcttttgtta

SEQ ID NO: 83 - DNA, 29 bp, Artificial Sequence
  PCR Primer
tttgttatttgttataccgatgtagtctc

SEQ ID NO: 84 - DNA, 27 bp, Artificial Sequence
  PCR Primer
tagctactcttctccaggtgtcattag

SEQ ID NO: 85 - DNA, 25 bp, Artificial Sequence
  PCR Primer
cctatgtccgagtttagcgagcttg

SEQ ID NO: 86 - DNA, 25 bp, Artificial Sequence
  PCR Primer
agaatgaactcagagttctctcttg

SEQ ID NO: 87 - DNA, 22 bp, Artificial Sequence
  PCR Primer
attctattgggtatgtcccctg

SEQ ID NO: 88 - DNA, 30 bp, Artificial Sequence
  PCR Primer
gtttttaattttgaagctcacctttaattg

SEQ ID NO: 89 - DNA, 26 bp, Artificial Sequence
  PCR Primer
attatgttctccagggaagaggttag

SEQ ID NO: 90 - DNA, 27 bp, Artificial Sequence
  PCR Primer
agaatcaatcatttagtgtggcaggag

SEQ ID NO: 91 - DNA, 25 bp, Artificial Sequence
  PCR Primer
taaaaactgcccgccatatttcgtc

SEQ ID NO: 92 - DNA, 708 bp, Zygosaccharomyces rouxii
ctgcagaaagccctaagatgctcctcccgttcacatgctccgaaccctttggaaaattctgtgcgcggcgctagcacgtaatgacccttgatgacaaactccaatggtatcaccctactgtcctctccccctcccctttttttccttctttctttccatctatttctgatctcctcccctcagcagatgtcccgaaaggtacagctgcgatacgggcagccactttttgacgtctcgcaacaggatcaccctgcacgacggggcacaataggattcccgttggcacggtgctggtgtatagccgccgagggtggggtataaagggctacatccttacccccacgcaggcgataacccgcatcatacaactgtcctcctcttccgctctcgccactagccgccgaaccattgctaccgcaatgacaccgtgtggtgatctcaagggaggatgtgtgggtgtgggacggaacttccactttttcctcagtaggtgcgatgccccctacaccgagcttccactaacgtgtttcagcggttgaaggcaatgggatcgcagaattatcgcagcttgttggtatataaagggagaagatatatggataagagacatgttctacttctgttctctctttctttttatcctatatcaccagaacaaatcaagttcgcattgattcatatcaaataaaaagtacatcacagataaca

SEQ ID NO: 93 - DNA, 21 bp, Artificial Sequence
  PCR Primer
tgcagaaagccctaagatgct

SEQ ID NO: 94 - DNA, 29 bp, Artificial Sequence
  PCR Primer
tgtctgtgatgtactttttatttgatatg

SEQ ID NO: 95 - DNA, 25 bp, Artificial Sequence
  PCR Primer
acgcaagagagaactctgagttcat